(19) United States
(12) Patent Application Publication
(10) Pub. No.: US 2006/0194244 A1
(43) Pub. Date: Aug. 31, 2006

(54) Uses of a chemokine receptor for inhibiting HIV-1 infection

(75) Inventors: Graham P. Allaway, Mohegan Lake, NY (US); Tatjana Dragic, Hartsdale, NY (US); Virginia M. Litwin, Fayetteville, NY (US); Paul J. Maddon, Elmsford, NY (US); John P. Moore, New York, NY (US); Alexandra Trkola, New York, NY (US)

Correspondence Address: COOPER & DUNHAM, LLP, 1185 AVENUE OF THE AMERICAS, NEW YORK, NY, 10036, US

(73) Assignees: Progenics Pharmaceuticals, Inc.; Aaron Diamonds AIDS, Research Centre (ADARC)

(21) Appl. No.: 11/400,497

(22) Filed: Apr. 7, 2006

Related U.S. Application Data
(63) Continuation of application No. 09/724,105, filed on Nov. 28, 2000.
Application No. 09/724,105 is a continuation of application No. 08/874,618, filed on Jun. 13, 1997, now abandoned.
(60) Provisional application No. 60/019,941, filed on Jun. 14, 1996.

(51) Int. Cl. C12Q 1/68; C12N 5/06; C12N 5/16

(52) U.S. Cl. 435/6; 435/345

(57) ABSTRACT

This invention provides a polypeptide comprising a fragment of a chemokine receptor capable of inhibiting HIV-1 infection. In an embodiment, the chemokine receptor is C-C CKR-5. In another embodiment, the fragment comprises at least one extracellular domain of the chemokine receptor C-C CKR-5. This invention further provides different uses of the chemokine receptor for inhibiting HIV-1 infection.

This application claims priority of U.S. Provisional Application Ser. No. 60/019,941, filed Jun. 14, 1996, the content of which is incorporated into this application by reference.

The invention described in this application was made with support under Grants Nos. Al35522, Al36057, Al36082 and Al38573 from the National Institutes of Health, U.S. Department of Health and Human Service. Accordingly, the United States Government has certain rights in this invention.

Throughout this application, various references are referred to by arabic numerals within parenthesis. Disclosures of these publications in their entireties are hereby incorporated by reference into this application to more fully describe the state of the art to which this invention pertains. Full bibliographic citation for these references may be found at the end of each series of experiments.

### BACKGROUND OF THE INVENTION

The replication of primary, non-syncytium-inducing (NSI) HIV-1 isolates in CD4⁺ T-cells is inhibited by the C-C β-chemokines MIP-1α, MIP-1β and RANTES (1, 2), but T-cell line-adapted (TCLA) or syncytium-inducing (SI) primary strains are insensitive (2, 3). The β-chemokines are small (8 kDa), related proteins active on cells of the lymphoid and monocyte lineage (4-8). Their receptors are members of the 7-membrane-spanning, G-protein-linked superfamily, one of which (the LESTR orphan receptor) has been identified as the second receptor for TCLA HIV-1 strains, and is now designated fusin (9). Fusin is not known to be a β-chemokine receptor (7-9).

### SUMMARY OF THE INVENTION

This invention provides a polypeptide having a sequence corresponding to the sequence of a portion of a chemokine receptor capable of inhibiting the fusion of HIV-1 to CD4⁺ cells and thus infection of the cells. In an embodiment, the chemokine receptor is C-C CKR-5. The C-C CKR-5 is also named as CCR5. In another embodiment, the polypeptide comprises amino acids having a sequence of at least one extracellular domain of C-C CKR-5.

In a preferred embodiment, the portion of a chemokine receptor comprises amino acid sequence MDYQVSSPIYDINYYTSEPCQKINVKQIAAR (SEQ ID NO: 5). In another preferred embodiment, the portion comprises amino acid sequence HYAAAQWDFGNTMCQ (SEQ ID NO: 6). In still another preferred embodiment, the portion comprises amino acid sequence RSQKEGLHYTCSSHFPYSQYQFWKNFQTLKIV (SEQ ID NO: 7). In a separate preferred embodiment, the portion comprises amino acid sequence QEFFGLNNCSSSNRLDQ (SEQ ID NO: 8). The portion of the chemokine receptor C-C CKR-5 may comprise one or more of the above sequences. The polypeptides may contain part of the above illustrated sequences and still be capable of inhibiting HIV-1 infection. The minimal number of amino acids sufficient to inhibit HIV-1 infection may be determined by the RET or infection assays as described below.

This invention also provides a pharmaceutical composition comprising effective amount of one or more of the above polypeptides and a pharmaceutically acceptable carrier.

This invention also provides a polypeptide having a sequence corresponding to that of a portion of an HIV-1 glycoprotein capable of specifically binding to the chemokine receptor C-C CKR-5.

This invention provides a pharmaceutical composition comprising effective amount of one of more polypeptides having a sequence corresponding to the sequence of a portion of an HIV-1 glycoprotein capable of specifically binding to the chemokine receptor C-C CKR-5 and a pharmaceutically acceptable carrier.

This invention provides an antibody or a portion of an antibody capable of binding to a chemokine receptor on a CD4⁺ cell and inhibiting HIV-1 infection of the cell.

This invention also provides a pharmaceutical composition comprising an effective amount of an antibody capable of binding to a chemokine receptor on a CD4⁺ cell and inhibiting HIV-1 infection of the cell and a pharmaceutically acceptable carrier.

This invention provides a method of treating an HIV-1 infected subject comprising administering to the subject the above polypeptides, antibody and pharmaceutical compositions.

This invention provides a method of reducing the likelihood of a subject from becoming infected by HIV-1 comprising administering to the subject the above pharmaceutical compositions.

This invention provides a method for inhibiting fusion of HIV-1 to CD4⁺ cells which comprises contacting CD4⁺ cells with a non-chemokine agent capable of binding to the chemokine receptor C-C CKR-5 in an amount and under conditions such that fusion of HIV-1 to the CD4⁺ cells is inhibited.

This invention provides a method for inhibiting HIV-1 infection of CD4⁺ cells which comprises contacting CD4⁺ cells with a non-chemokine agent capable of binding to the chemokine receptor C-C CKR-5 in an amount and under conditions such that fusion of HIV-1 to the CD4⁺ cells is inhibited, thereby inhibiting HIV-1 infection of the cells.

This invention provides a non-chemokine agent capable of binding to the chemokine receptor C-C CKR-5 and inhibiting HIV-1 infection.

This invention provides a molecule capable of binding to the chemokine receptor C-C CKR-5 and inhibiting HIV-1 infection comprising a non-chemokine agent linked to a ligand capable of binding to a cell surface receptor of the CD4⁺ cells other than the chemokine receptor such that the binding of the non-chemokine agent to the chemokine receptor does not prevent the binding of the ligand to the other receptor.

This invention provides a pharmaceutical composition comprising an amount of the above molecules effective to inhibit fusion of HIV-1 to CD4⁺ cells and a pharmaceutically acceptable carrier.

This invention also provides a molecule capable of binding to the chemokine receptor C-C CKR-5 and inhibiting fusion of HIV-1 to CD4⁺ cells comprising a non-chemokine agent linked to a compound capable of increasing the in vivo half-life of the non-chemokine agent.

This invention further provides a pharmaceutical composition comprising an amount of the molecule capable of binding to the chemokine receptor C-C CKR-5 and inhibiting HIV-1 infection comprising a non-chemokine agent linked to a molecule encoding a sufficient portion of the CD4 molecule to permit binding the HIV-1 envelope glycoprotein.

This invention further provides a transgenic nonhuman animal which comprises an isolated DNA molecule encoding the chemokine receptor C-C CKR-5 and an isolated DNA molecule encoding fusin. In an embodiment, this transgenic nonhuman animal further comprises an isolated DNA molecule encoding the full-length or portion of the CD4 molecule sufficient for binding the HIV-1 envelope glycoprotein.

This invention also provides transformed cells which comprise an isolated nucleic acid molecule encoding the chemokine receptor C-C CKR-5.

Finally, his invention provides an agent capable of inhibiting HIV-1 infection and capable of binding to a chemokine receptor without substantially affecting the said chemokine receptor's capability to bind to chemokines.

### DESCRIPTION OF THE FIGURES

FIG. 1: Specificity, time course and stage of β-chemokine inhibition of HIV-1 replication

(1A) PM1 cells (1×10⁶) were preincubated with RANTES+MIP-1α+MIP-1β (R/Mα/Mβ; 100 ng/ml of each) for 24 h (−24 h) or 2 h (−2 h), then washed twice with phosphate buffered saline (PBS). HIV-1 (BaL env-complemented) virus (50 ng of p24; see legend to Table 1) was added for 2 h, then the cells were washed and incubated for 48 h before measurement of luciferase activity in cell lysates as described previously (10, 11). Alternatively, virus and R/Mα/Mβ were added simultaneously to cells, and at the indicated time points (1 h, 3 h, etc) the cells were washed twice in PBS, resuspended in culture medium and incubated for 48 h prior to luciferase assay. Time 0 represents the positive control, to which no β-chemokines were added. +2 h represents the mixture of virus with cells for 2 h prior to washing twice in PBS, addition of R/Mα/Mβ and continuation of the culture for a further 48 h before luciferase assay.

(1B) PM1 cells (1×10⁶) were infected with HIV-1 (500 pg p 24) grown in CEM cells (NL4/3; lanes 1-4) or macrophages (ADA; lanes 5-8), in the presence of 500 ng/ml of RANTES (lanes 1 and 5) or MIP-1β (lanes 2 and 6), or with no β-chemokine (lanes 4 and 8). Lanes 3 and 7 are negative controls (no virus). All viral stocks used for the PCR assay were treated with DNAse for 30 min at 37° C., and tested for DNA contamination before use. After 2 h, the cells were washed and resuspended in medium containing the same β-chemokines for a further 8 h. DNA was then extracted from infected cells using a DNA/RNA isolation kit (US Biochemicals). First round nested PCR was performed with primers: U3+, 5′-CAAGGCTACTTCCCTGATTGGCAGAACTACACACCAGG-3′ (SEQ ID NO:1) preGag, 5′-AGCAAGCCGAGTCCTGCGTCGAGAG-3′ (SEQ ID NO:2) and the second round with primers: LTR-test, 5′-GGGACTTTCCGCTGGGGACTTTC 3′ (SEQ ID NO:3) LRC2,5′-CCTGTTCGGGCGCCACTGCTAGAGATTTTCCAC 3′ (SEQ ID NO:4) in a Perkin Elmer 2400 cycler with the following amplification cycles: 94° C. for 5 min, 35 cycles of 94° C. for 30 s, 55° C. for 30 s, 72° C. for 30 s, 72° C. for 7 min. M indicates 1 kb DNA ladder; 1, 10, 100, 1000 indicate number of reference plasmid (pAD8) copies. The assay can detect 100 copies of reverse transcripts.

FIG. 2: HIV-1 env-mediated membrane fusion of cells transiently expressing C-C CKR-5

Membrane fusion mediated by β-chemokine receptors expressed in HeLa cells was demonstrated as follows: Cells were transfected with control plasmid pcDNA3.1 or plasmid pcDNA3.1-CKR constructs using lipofectin (Gibco BRL). The pcDNA3.1 plasmid carries a T7-polymerase promoter and transient expression of β-chemokine receptors was boosted by infecting cells with 1×10⁷ pfu of vaccinia encoding the T7-polymerase (vFT7.3) 4 h post-lipofection (9). Cells were then cultured overnight in R18-containing media and were tested for their ability to fuse with HeLa-JR-FL cells (filled columns) or HeLa-BRU cells (hatched column) in the RET assay. The % RET with control HeLa cells was between 3% and 4% irrespective of the transfected plasmid.

compound capable of increasing the in vivo half-life of the non-chemokine agent effective to inhibit fusion of HIV-1 to CD4⁺ cells and a pharmaceutically acceptable carrier.

This invention provides a method for reducing the likelihood of HIV-1 infection in a subject comprising administering the above pharmaceutical compositions to the subject.

This invention provides a method for treating HIV-1 infection in a subject comprising administering the above pharmaceutical compositions to the subject.

This invention provides a method for determining whether a non-chemokine agent is capable of inhibiting the fusion of HIV-1 to a CD4⁺, C-C CKR-5⁺ cell which comprises: (a) contacting a CD4⁺, C-C CKR-5⁺ cell, which is labeled with a first dye, with a cell expressing an appropriate HIV-1 envelope glycoprotein on its surface, which is labeled with a second dye, in the presence of excess of the agent under conditions permitting the fusion of the CD4⁺ and C-C CKR-5⁺ cell to the cell expressing the HIV-1 envelope glycoprotein on its surface in the absence of an agent known to inhibit fusion of HIV-1 to CD4⁺, C-C CKR-5⁺ cell, the first and second dyes being selected so as to allow resonance energy transfer between the dyes; (b) exposing the product of step (a) to conditions which would result in resonance energy transfer if fusion has occurred; and (c) determining whether there is resonance energy transfer, the absence or reduction of transfer indicating that the agent is capable of inhibiting fusion of HIV-1 to CD4⁺ and C-C CKR-5⁺ cells.

This invention also provides a transgenic nonhuman animal which comprises an isolated DNA molecule encoding the chemokine receptor C-C CKR-5. In an embodiment, this transgenic nonhuman animal further comprises an isolated DNA

FIG. 3: CD4-dependent competition between gp120 and MIP-1β for CCR-5 binding.

(3A) JR-FL gp120 (filled squares), LAI gp120 (filled triangles), JR-FL-ΔV3 gp120 (open squares or LAI-ΔV3 gp120 (open trangles) was added to activated CD4⁺ T cells and the extent of specific ¹²⁵I-MIP-1β binding determined. Data shown are the means of three independent experiments, each performed in duplicate.

(3B) JR-FL gp120 (2 μg ml⁻¹) and ¹²⁵I-MIP-1β (0.1 μM) were added to activated CD4⁺ T cells in the presence of the monoclonal antibody Q4120 (filled circles) or sCD4 (filled squares) at the concentrations indicated. The extent of specific ¹²⁵I-MIP-1β binding was determined, and the percentage inhibition of the gp120 competitive effect was calculated for each antibody concentration (none present is 0% inhibition). The experiment shown was one of two performed, each yielding similar results.

FIG. 4: Mutagenesis of the predicted four extracellular domains of CCR5

The amino acid sequences of the human CCR5 amino terminus (Nt) and three extracellular loops (ECL 1-3) are indicated (19, 20 of the Third Series of Experiments). The polarity (+ or −) of charged residues is indicated below the main sequences, as are the identities of residues which differ in murine CCR5. Human CCR5 residues with negatively (white squares) and positively charged side chains (black squares), and residues whose charge differed in murine CCR5 (white circles), were all modified to alanine by PCR or site-directed mutagenesis. Fidelity was confirmed by sequencing both strands of the entire CCR5 coding region. In some cases, double mutants K171A/E172A, K191A/N192A and R274A/D276A were made, to preserve the overall net charge of their domain. The Nt double and triple mutants D2A/D11A and D2A/D11A/E18A were based on initial results with single residue mutants.

FIG. 5: HIV-1 co-receptor function of CCR5 mutants

Substitutions in (A) negatively charged residues; (B) positively charged residues; (C) selected murine residues differing from the human sequence were tested for their effects on HIV-1 entry. U87MG-CD4 cells were transiently lipofected with CCR5 mutants, then infected with NLluc/ADA (dark hatched bars), NLluc/JR-FL (light hatched bars) or NLluc/DH123 (white bars) luciferase-expressing chimeric viruses (1, 2 of the Third Series of Experiments). Luciferase activity (luc c.p.s.) was measured 72 h post-infection (1, 2) and standardized for lipofection efficiency and receptor expression levels. The co-receptor activity of each mutant designated on the x-axis is expressed as a percentage of the wild-type co-receptor activity (100%), and is the mean±s.d. of three independent experiments each performed in quadruplicate. (*) indicates that the amino acid is also different in murine CCR5. Similar results (not shown) were obtained with SCL-1-CD4 cells.

FIG. 6: Membrane fusion activity of CCR5 Nt mutants

HeLa-CD4 cells were lipofected with the Nt mutants indicated (or the pcDNA3.1 negative control plasmid), and tested 48 h later for their ability to fuse with HeLa cells expressing the JR-FL env gene (black bars) (1, 18 of the Third Series of Experiments). The vFT7pol system was used to enhance co-receptor expression (hatched bars) (1, 4, 5, 13 of the Third Series of Experiments). The extent of cell-cell fusion was determined using the RET assay (1.18 of the Third Series of Experiments). The % RET values shown are the means±s.d. of three independent experiments, each performed in duplicate.

FIG. 7: Competition between gp120 and CCR5 MAb 2D7 for CCR5 binding

HeLa cells co-transfected with CD4 and either wild-type or mutant CCR5, and infected with vFT7pol to enhance receptor expression, were pre-incubated with or without 10 μg/ml gp120 (JR-FL) (7) before addition of 2 ng/ml of the PE-labeled 2D7 MAb (23, 24 of the Third Series of Experiments) and FACS analysis to determine mean fluorescence intensity (m.f.i.). Inhibition of 2D7-PE binding is presented as [1−(m.f.i. with gp120/m.f.i. without gp120)]×100%, and is the mean±s.d. of three independent experiments.

FIG. 8. Flow cytometric analysis of the binding of sCD4-gp120 complexes to (B)CCR5⁻ and (B)CCR5⁺ L1.2 cells, a murine pre-B lymphoma line

Cells are incubated for 15 min. with equimolar (˜100 nM) mixtures of sCD4 and biotinylated HIV-1_{JR-FL} gp120 and then stained with a streptavidin-phycoerythrin conjugate, fixed with 2% paraformaldehyde, and analyzed by FACS. Cell number is plotted on the y-axis.

FIG. 9. Inhibition of binding of HIV-1_{JR-FL} gp120, complexed with sCD4, to butyrate-treated L1.2 CCR5⁺ cells

The inhibitors were the CC chemokines MIP-1β or RANTES at the concentrations indicated on the x axis.

FIG. 10 Inhibition of HIV-1 envelope-mediated cell fusion by the bicyclam JM3100

The inhibition was measured using the RET assay, with the cell combinations indicated.

### DETAILED DESCRIPTION OF THE INVENTION

This invention provides a polypeptide having a sequence corresponding to the sequence of a portion of a chemokine receptor capable of inhibiting the fusion of HIV-1 to CD4⁺ cells and thus infection of the cell. In an embodiment, the chemokine receptor is C-C CKR-5 (CCR5). In another embodiment, the fragment comprises at least one extracellular domain of the chemokine receptor C-C CKR-5. In a further embodiment, the extracellular domain is the second extracellular loop of CCR5.

In a separate embodiment, the chemokine receptor is CCR3 or CKR-2b (31, 32).

The sequence of a portion of the chemokine receptor includes the original amino acids or modified amino acids from the receptor, their derivatives and analogues. Such sequence should retain the ability to inhibit HIV-1 infection. Sequences of fusin are also included.

In a preferred embodiment, the portion of a chemokine receptor comprises amino acid sequence MDYQVSSPIYDINYYTSEPCQKINVKQIAAR (SEQ ID NO: 5). In another preferred embodiment, the portion comprises amino acid sequence HYAAAQWDFGNTMCQ (SEQ ID NO: 6). In still another preferred embodiment, the portion comprises amino acid sequence RSQKEGLHYTCSSHFPYSQYQFWKNFQTLKIV (SEQ ID NO: 7). In a separate preferred embodiment, the portion comprises amino acid sequence QEFFGLNNCSSSNRLDQ (SEQ ID NO: 8). The portion of the chemokine receptor C-C CKR-5 may comprise one or more of the above sequences. The polypeptides may contain part of the above illustrated sequences and still be capable of inhibiting HIV-1 infection. The minimal number of amino acids sufficient to inhibit HIV-1 infection may be determined by the RET or infection assays as described below.

The polypeptides described above may be fusion molecules such that the fragments are linked to other molecules. In an embodiment, the molecule is a CD4-based molecule. CD4-based molecules are known in the art and described in Allaway et al. (1996), Patent Cooperation Treaty Application No. PCT/US95/08805, publication no. WO 96/02575, the content of which is incorporated by reference into this application. In another embodiment, the polypeptide contains multiple units of one or more portions of a chemokine receptor. In a preferred embodiment, the polypeptide contains sequences corresponding to multiple units of one or more extracellular domains of the chemokine receptor C-C CKR-5.

This invention also provides a pharmaceutical composition comprising effective amount of the above polypeptide and a pharmaceutically acceptable carrier.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents.

This invention also provides a polypeptide having a sequence corresponding to that of a portion of an HIV-1 envelope glycoprotein capable of specifically binding to the chemoreceptor C-C CKR-5. Such a sequence may be identified by routine experiments. For example, overlapping synthetic peptides representing fragments of gp120 or gp41 can be tested in the RET assay for their ability to inhibit fusion between cells expressing the envelope glycoprotein of HIV-1 clinical isolates and cells expressing CD4 and C-C CKR-5. Peptides inhibiting fusion in this assay are also screened in the RET assay for ability to inhibit fusion mediated by the envelope glycoprotein of HIV-1 laboratory-adapted-strains and peptides which are inhibitory in this later assay are discarded. As an alternative method, the peptides can be tested for their ability to compete with chemokines for binding to cell expressing C-C CKR-5.

This invention provides a pharmaceutical composition comprising effective amount of the polypeptide comprising a fragment of HIV-1 glycoprotein capable of specifically binding to the chemokine receptor C-C CKR-5 and a pharmaceutically acceptable carrier.

This invention provides an antibody or a portion of an antibody thereof capable of binding to a chemokine receptor on a CD4⁺ cell and inhibiting HIV-1 infection of the cell.

This invention also provides a pharmaceutical composition comprising effective amount of antibody capable of binding to a chemokine receptor and inhibiting HIV-1 infection and a pharmaceutically acceptable carrier.

This invention provides a method of treating an HIV-1 infected subject comprising administering to the subject the above pharmaceutical compositions.

This invention provides a method of reducing the likelihood of a subject from becoming infected by HIV-1 comprising administering to the subject the above pharmaceutical compositions.

This invention provides a method for inhibiting fusion of HIV-1 to CD4⁺ cells which comprises contacting CD4⁺ cells with a non-chemokine agent capable of binding to the chemokine receptor C-C CKR-5 in an amount and under conditions such that fusion of HIV-1 to the CD4⁺ cells is inhibited.

This invention provides a method for inhibiting HIV-1 infection of CD4⁺ cells which comprises contacting CD4⁺ cells with a non-chemokine agent capable of binding to the chemokine receptor C-C CKR-5 in an amount and under conditions such that fusion of HIV-1 to the CD4⁺ cells is inhibited, thereby inhibiting HIV-1 infection.

The non-chemokine agents of this invention are capable of binding to chemokine receptors and inhibiting fusion of HIV-1 to CD4⁺ cells. The non-chemokine agents include, but are not limited to, chemokine fragments and chemokine derivatives and analogues, but do not include naturally occurring chemokines.

In an embodiment, the non-chemokine agent is an oligopeptide. In another embodiment, the non-chemokine agent is a polypeptide. In still another embodiment, the non-chemokine agent is a nonpeptidyl agent.

This invention provides a non-chemokine agent capable of binding to the chemokine receptor C-C CKR-5 and inhibiting fusion of HIV-1 to CD4⁺ cells.

This invention provides a molecule capable of binding to the chemokine receptor C-C CKR-5 and inhibiting fusion of HIV-1 to CD4⁺ cells comprising a non-chemokine agent linked to a ligand capable of binding to a cell surface receptor of the CD4⁺ cells other than the chemokine receptor such that the binding of the non-chemokine agent to the chemokine receptor does not prevent the binding of the ligand to the other receptor. In an embodiment, the cell surface receptor is CD4. In another embodiment, the ligand comprises an antibody or a portion of an antibody.

This invention provides a pharmaceutical composition comprising an amount of the above molecule effective to inhibit fusion of HIV-1 to CD4⁺ cells and a pharmaceutically acceptable carrier.

This invention also provides a molecule capable of binding to the chemokine receptor C-C CKR-5 and inhibiting fusion of HIV-1 to CD4⁺ cells comprising a non-chemokine agent linked to a compound capable of increasing the in vivo half-life of the non-chemokine agent. In an embodiment, the compound is polyethylene glycol.

This invention further provides a pharmaceutical composition comprising an amount of the molecule capable of binding to the chemokine receptor C-C CKR-5 and inhibiting fusion of HIV-1 to CD4⁺ cells comprising a non-chemokine agent linked to a compound capable of increasing the in vivo half-life of the non-chemokine agent effective to inhibit fusion of HIV-1 to CD4⁺ cells and a pharmaceutically acceptable carrier.

This invention provides a method for reducing the likelihood of HIV-1 infection in a subject comprising administering the above pharmaceutical compositions to the subject.

This invention provides a method for treating HIV-1 infection in a subject comprising administering the above pharmaceutical compositions to the subject.

This invention provides a method for determining whether a non-chemokine agent is capable of inhibiting the fusion of HIV-1 to a CD4⁺, C-C CKR-5⁺ cell which comprises: (a) contacting a CD4⁺, C-C CKR-5⁺ cell, which is labeled with a first dye, with a cell expressing an appropriate HIV-1 envelope glycoprotein on its surface, which is labeled with a second dye, in the presence of excess of the agent under conditions permitting the fusion of the CD4⁺ and C-C CKR-5⁺ cell to the cell expressing the HIV-1 envelope glycoprotein on its surface in the absence of an agent known to inhibit fusion of HIV-1 to CD4⁺, C-C CKR-5⁺ cell, the first and second dyes being selected so as to allow resonance energy transfer between the dyes; (b) exposing the product of step (a) to conditions which would result in resonance energy transfer if fusion has occurred; and (c) determining whether there is resonance energy transfer, the absence or reduction of transfer indicating that the agent is capable of inhibiting fusion of HIV-1 to CD4⁺ and C-C CKR-5⁺ cells. In an embodiment, the agent is an oligopeptide. In another embodiment, the agent is a polypeptide. In still another embodiment, the agent is a nonpeptidyl agent. In a further embodiment, the CD4⁺ cell is a PM1 cell. In a separate embodiment, the cell expressing the HIV-1 envelope glycoprotein is a HeLa cell expressing HIV-1_{JR-FL} gp120/gp41.

This invention also provides a transgenic nonhuman animal which comprises an isolated DNA molecule encoding the chemokine receptor C-C CKR-5. In an embodiment, this transgenic nonhuman animal further comprises an isolated DNA molecule encoding a sufficient portion of the CD4 molecule to permit binding the HIV-1 envelope glycoprotein.

This invention further provides a transgenic nonhuman animal which comprises an isolated DNA molecule encoding the chemokine receptor C-C CKR-5 and an isolated DNA molecule encoding fusin. In an embodiment, this transgenic nonhuman animal further comprises an isolated DNA molecule encoding a sufficient portion of the CD4 molecule to permit binding the HIV-1 envelope glycoprotein.

One means available for producing a transgenic animal, with a mouse as an example, is as follows: Female mice are mated, and the resulting fertilized eggs are dissected out of their oviducts. The eggs are stored in an appropriate medium such as M2 medium (Hogan B. et al. Manipulating the Mouse Embryo, A Laboratory Manual, Cold Spring Harbor Laboratory (1986)). DNA or cDNA encoding the C-C CKR-5 chemokine receptor or CD4 is purified from a vector by methods well known in the art. Inducible promoters may be fused with the coding region of the DNA to provide an experimental means to regulate expression of the trans-gene. Alternatively or in addition, tissue specific regulatory elements may be fused with the coding region to permit tissue-specific expression of the trans-gene. The DNA, in an appropriately buffered solution, is put into a microinjection needle (which may be made from capillary tubing using a pipet puller) and the egg to be injected is put in a depression slide. The needle is inserted into the pronucleus of the egg, and the DNA solution is injected.

The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only method for inserting DNA into the egg cell, and is used here only for exemplary purposes.

This invention provides transformed cells which comprise an isolated nucleic acid molecule encoding the chemokine receptor C-C CKR-5.

This invention also provides an agent capable of inhibiting HIV-1 infection and capable of binding to a chemokine receptor without substantially affecting the said chemokine receptor's capability to bind to chemokines.

As used herein, the term “without substantially affecting” mean that after the binding of the agent to the chemokine receptor, the chemokine receptor should still be able to bind to chemokines. Under some conditions, following binding of an agent to a chemokine receptor, a higher concentration of the chemokine is required to achieve the degree of binding observed if the chemokine receptor had not been bound to the agent. In a preferred embodiment of this agent, the chemokine concentration required to achieve the same binding is two fold. In another embodiment, the chemokine concentration is ten fold.

In a preferred embodiment of this invention, the chemokine receptor is CCR5. In another embodiment, the chemokine receptor is CXCR4, CCR3 or CCR-2b.

This agent may be an oligopeptide, a nonpeptidyl agent or a polypeptide. Alternatively, this agent can be an antibody or a portion of an antibody.

This invention further provides a pharmaceutical composition comprising an amount of the above agent effective to inhibit fusion of HIV-1 to CD4⁺ cells and a pharmaceutically acceptable carrier.

This invention provides a method for inhibiting HIV-1 infection of CD4⁺ cells which comprises contacting such CD4+ cells with an agent capable of inhibiting HIV-1 infection and capable of binding to a chemokine receptor without substantially affecting the said chemokine receptor's capability to bind to chemokines.

This invention also provides a molecule capable of binding to the chemokine receptor CCR5 and inhibiting fusion of HIV-1 to CD4⁺ cells without substantially affecting the said chemokine receptor's capability to bind to chemokines linked to a compound capable of increasing the in vivo half-life of the non-chemokine agent. In an embodiment, the compound is polyethylene glycol.

This invention provides a pharmaceutical composition comprising an amount of the above molecule effective to inhibit HIV-1 infection and a pharmaceutically acceptable carrier.

This invention provides a method for reducing the likelihood of HIV-1 infection in a subject comprising administering the above pharmaceutical composition to the subject.

This invention provides a method for treating HIV-1 infection in a subject comprising administering the above pharmaceutical composition to the subject.

This invention provides a method for determining whether an agent is capable of inhibiting HIV-1 infection comprising steps of: (a) fixing a chemokine receptor on a solid matrix; (b) contacting the agent with the fixed chemokine receptor under conditions permitting the binding of the agent to the chemokine receptor; (c) removing the unbound agent; (d) contacting the fixed chemokine receptor resulting in step (c) with a gp120 in the presence of CD4 under conditions permitting the binding of the gp120/CD4 complex and the chemokine receptor in the absence of the agent; (e) measuring the amount of bound gp120/CD4 complex; and (f) comparing the amount determined in step (d) with the amount determined in the absence of the agent, a decrease of the amount indicating that the agent is capable of inhibiting HIV-1 infection.

This invention also provides a method for determining whether an agent is capable of inhibiting HIV-1 infection comprising steps of: (a) fixing a chemokine receptor on a solid matrix; (b) contacting the agent with the fixed chemokine receptor; (c) contacting the mixture in step (b) with a gp120 in the presence of CD4 under conditions permitting the binding of the gp120/CD4 complex and the chemokine receptor in the absence of the agent; (d) measuring the amount of bound gp120/CD4 complex; and (e) comparing the amount determined in step (d) with the amount determined in the absence of the agent, a decrease of the amount indicating that the agent is capable of inhibiting HIV-1 infection.

This invention also provides a method for determining whether an agent is capable of inhibiting HIV-1 infection comprising steps of: (a) fixing a gp120/CD4 complex on a solid matrix; (b) contacting the agent with the fixed gp120/CD4 complex under conditions permitting the binding of the agent to the gp120/CD4 complex; (c) removing unbound agent; (d) contacting the fixed gp120/CD4 complex resulting from step (c) with a chemokine receptor under conditions permitting the binding of the gp120/CD4 complex and the chemokine receptor in the absence of the agent; (e) measuring the amount of bound chemokine receptor; and (f) comparing the amount determined in step (e) with the amount determined in the absence of the agent, a decrease of the amount indicating that the agent is capable of inhibiting HIV-1 infection.

This invention provides a method for determining whether an agent is capable of inhibiting HIV-1 infection comprising steps of: (a) fixing a gp120/CD4 on a solid matrix; (b) contacting the agent with the fixed gp120/CD4 complex; (c) contacting the mixture in step (b) with a chemokine receptor under conditions permitting the binding of the gp120/CD4 complex and the chemokine receptor in the absence of the agent; (d) measuring the amount of bound chemokine receptor; (e) comparing the amount determined in step (d) with the amount determined in the absence of the agent, a decrease of the amount indicating that the agent is capable of inhibiting HIV-1 infection.

As used in these assays, CD4 include soluble CD4, fragment of CD4 or polypeptides incorporating the gp120 binding site of CD4 capable of binding gp120 and enabling the binding of gp120 to the appropriate chemokine receptor.

As used in these assays, gp120 is the gp120 from an appropriate strain of HIV-1. For example, gp120 from the macrophage tropic clinical isolate HIV-1_{JR-FL} will bind to the chemokine receptor CCR5, whereas gp120 from the laboratory adapted T-tropic strain HIV-1 _{LAI} will bind to the chemokine receptor CXCR4.

In a preferred embodiment of the above methods, the CD4 is a soluble CD4. The chemokine receptor which may be used in the above assay includes CCR5, CXCR4, CCR3 and CCR-2b.

In an embodiment, the chemokine receptor is expressed on a cell. In a preferred embodiment, the cell is a L1.2 cell. In a separate embodiment, the chemokine receptor is purified and reconstituted in liposomes. Such chemokine receptor embedded in the lipid bilayer of liposomes retains the gp120 binding activity of the receptor.

The gp120, CD4 or both may be labelled with a detectable marker in the above assays. Markers including radioisotope or enzymes such as horse radish peroxidase may be used in this invention.

In an embodiment, the gp120 or CD4 or the chemokine receptor is labelled with biotin. In a further embodiment, the biotinylated gp120, or CD4 or the chemokine receptor is detected by: (i) incubating with streptavidin-phycoerythrin, (ii) washing the incubated mixture resulting from step (i), and (iii) measuring the amount of bound gp120 using a plate reader, exciting at 530 nm, reading emission at 590 nm.

This invention also provides an agent determined to be capable of inhibiting HIV-1 infection by the above methods, which is previously unknown.

This invention also provides a pharmaceutical composition comprising the agent determined to be capable of inhibiting HIV-1 infection by the above methods and a pharmaceutically acceptable carrier. In an embodiment, the agent is an oligopeptide. In another embodiment, the agent is a polypeptide. In a still another embodiment, the agent is a nonpeptidyl agent.

This invention also provides a molecule capable of binding to the chemokine receptor CCR5 and inhibiting fusion of HIV-1 to CD4⁺ cells comprising the above determined agent linked to a compound capable of increasing the in vivo half-life of the non-chemokine agent. In an embodiment, the compound is polyethylene glycol. This invention also provides a pharmaceutical composition comprising an amount of the above molecule effective to inhibit HIV-1 infection and a pharmaceutically acceptable carrier.

This invention provides a method for reducing the likelihood of HIV-1 infection in a subject comprising administering the above pharmaceutical compositions to the subject.

This invention provides a method for treating HIV-1 infection in a subject comprising administering the above pharmaceutical composition to the subject.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Experimental Details

To study how β-chemokines inhibit HIV-1 replication, a virus entry assay based on single-cycle infection by an env-deficient virus, NL4/3Δenv (which also carries the luciferase reporter gene), complemented by envelope glycoproteins expressed in trans was used (10, 11). Various env-complemented viruses were tested in PM1 cells, a variant of HUT-78 that has the unique ability to support replication of primary and TCLA HIV-1 strains, allowing comparison of envelope glycoprotein functions against a common cellular background (2, 12). MIP-1α, MIP-1β and RANTES are most active against HIV-1 in combination (2, 3), and strongly inhibited infection of PM1 cells by complemented viruses whose envelopes are derived from the NSI primary strains ADA and BaL (Table 1a).

TABLE 1
Inhibition of HIV-1 entry in PM1 cells
and CD4⁺ T-cells by β-chemokines

| | % luciferase activity | | | | |
|---|---|---|---|---|---|
| a) | BaL | ADA | NL4/3 | HxB2 | MuLV |
| PM1 cells | | | | | |
| control without virus | 2 | 2 | 2 | 5 | 3 |
| control with virus | 100 | 100 | 100 | 100 | 100 |
| +R/Mα/Mβ (50/50/50) | 2 | 3 | 92 | 117 | 100 |
| +RANTES (100) | 1 | 1 | nd | nd | nd |
| +MIP-1α(100) | 54 | 54 | nd | nd | nd |
| +MIP-1β (100) | 1 | 6 | nd | nd | nd |
| +MCP-1 (100) | 46 | 50 | nd | nd | nd |
| +MCP-2 (100) | 28 | 26 | nd | nd | nd |
| +MCP-3 (100) | 58 | 46 | nd | nd | nd |

| b) | JR-FL | HxB2 | MuLV |
|---|---|---|---|
| LW4 CD4⁺ T-cells | | | |
| control without virus | 1 | 1 | 1 |
| control with virus | 100 | 100 | 100 |
| +R/Mα/Mβ (200/200/200) | 14 | 68 | nd |
| LW5 CD4⁺ T-cells | | | |
| control without virus | 1 | 1 | 1 |
| control with virus | 100 | 100 | 100 |
| +R/Mα/Mβ (200/200/200) | 15 | 73 | nd |

Table 1 Legend:

PM1 cells were cultured as described by Lusso et al (12). Ficoll/hypaque-isolated PBMC from laboratory workers (LW) stimulated with PHA for 72 h before depletion of CD8+ Lymphocytes with anti-CD8 immunomagnetic beads (DYNAL, Great Neck, N.Y.). CD4+ Lymphocytes were maintained in culture medium containing interleukin-2 (100 U/ml; Hofmann LaRoche, Nutley, N.J.), as described previously (3). Target cells (1-2×10⁵) were infected with supernatants (10-50 ng of HIV-1 p24) from 293-cells co-transfected with an NL4/3Δenv-luciferase vector and a HIV-1 env-expressing vector (10, 11). β-Chemokines (R & D Systems, Minneapolis) were added to the target cells simultaneously with virus, at the final concentrations (ng/ml) indicated in parentheses in the first column. The β-chemokine concentration range was selected based on prior studies (2, 3). After 2 h, the cells were washed twice with PBS, resuspended in β-chemokine-containing media and maintained for 48-96 h. Luciferase activity in cell lysates was measured as described previously (10, 11). The values indicated represent luciferase activity (cpm)/ng p24/mg protein, expressed relative to that in virus-control cultures lacking β-chemokines (100%), and are the means of duplicate or sextuplicate determinations. nd, not done. R/Mα/Mβ, RANTES+MIP-1α+MIP-1β.

RANTES and MIP-1β were strongly active when added individually, while other β-chemokines—MlP-1α, MCP-1, MCP-2 and MCP-3 (refs. 13-15)—were weaker inhibitors (Table 1a). However, MIP-1α, MIP-1β and RANTES, in combination, did not inhibit infection of PM1 cells by the TCLA strains NL4/3 and HxB2, or by the amphotropic murine leukemia virus (MuLV-Ampho) pseudotype (Table 1a). Thus, phenotypic characteristics of the HIV-1 envelope glycoproteins influence their sensitivity to β-chemokines in a virus entry assay.

The env-complementation assay was used to assess HIV-1 entry into CD4+ T-cells from two control individuals (LW4 and LW5). MIP-1α, MIP-1β and RANTES strongly inhibited infection by the NSI primary strain JR-FL infection of LW4's and LW5's CD4⁺ T-cells, and weakly reduced HxB2 infection of LW cells (Table 1b), suggesting that there may be some overlap in receptor usage on activated CD4⁺ T-cells by different virus strains. BaL env-mediated replication in normal PBL was also inhibited by MIP-1α, MIP-1β and RANTES, albeit with significant inter-donor variation in sensitivity (data not shown).

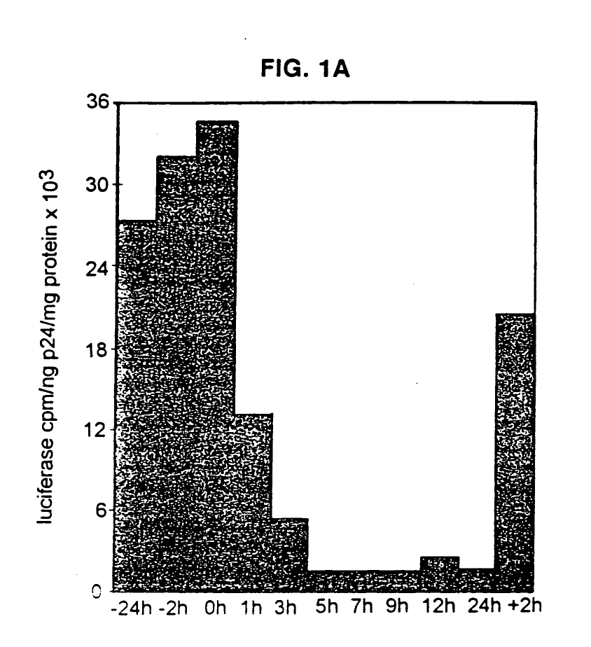

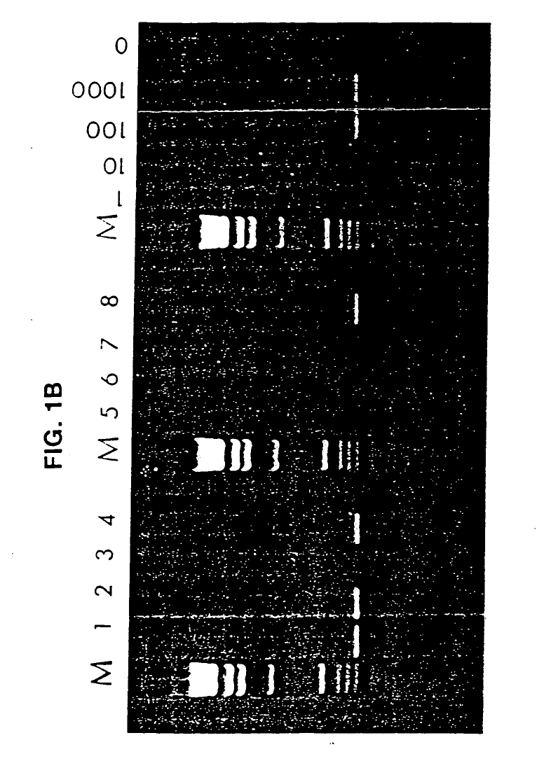

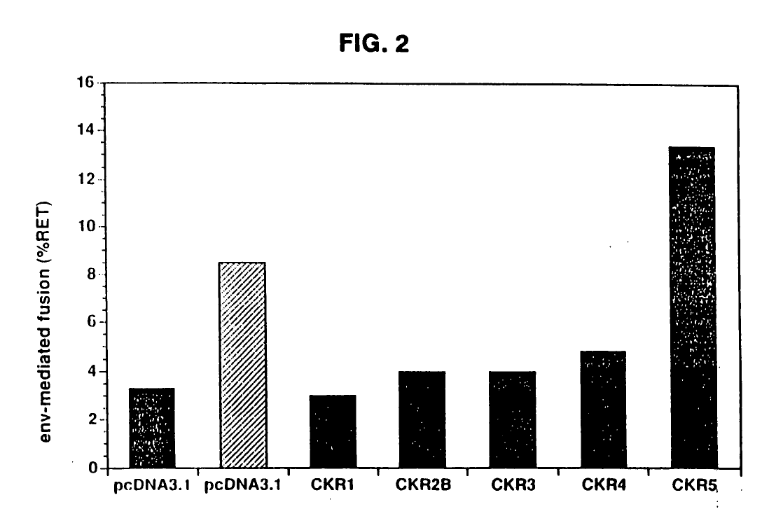

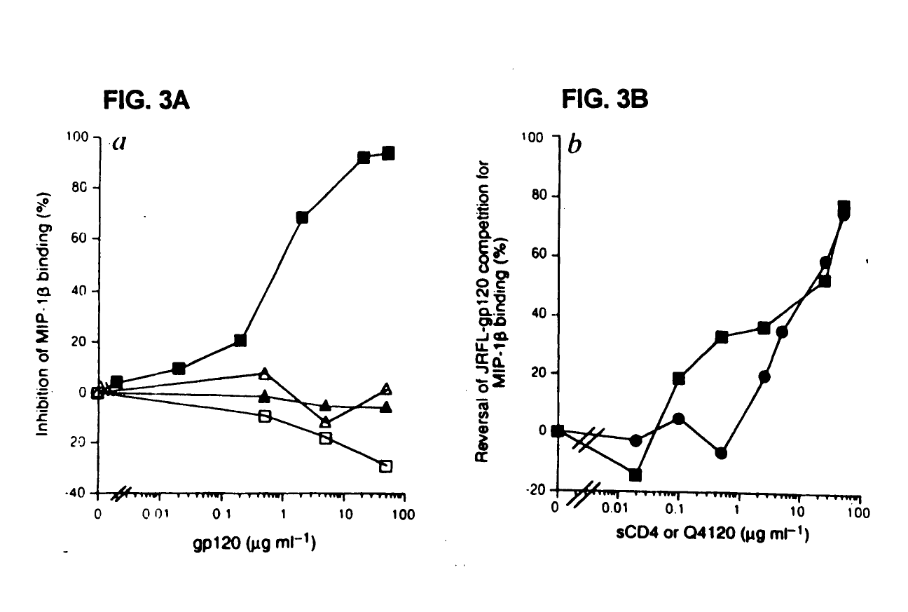

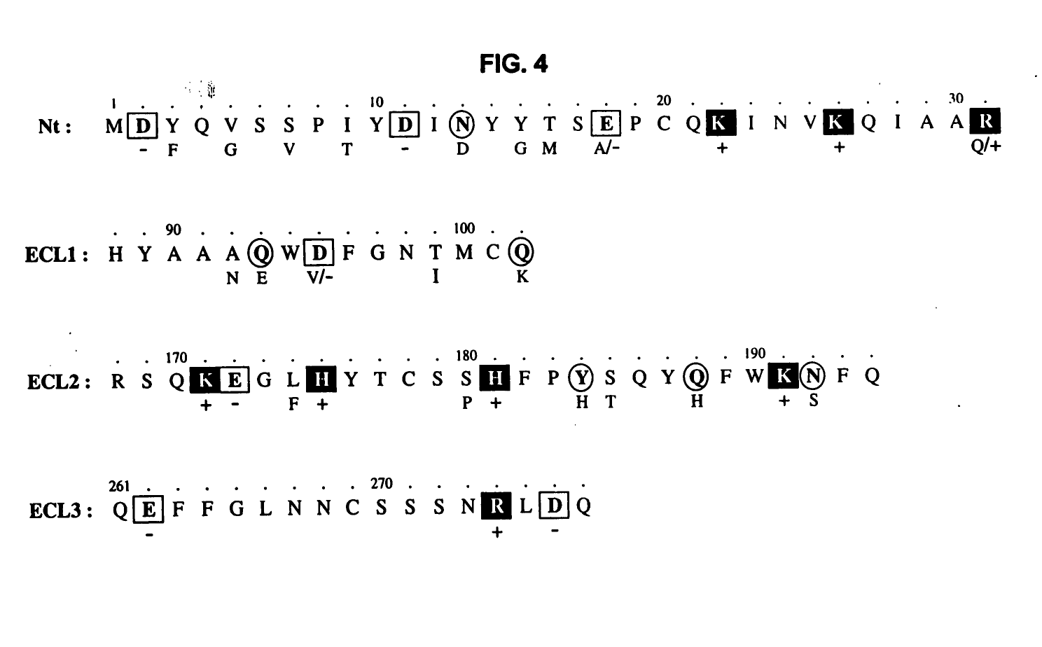

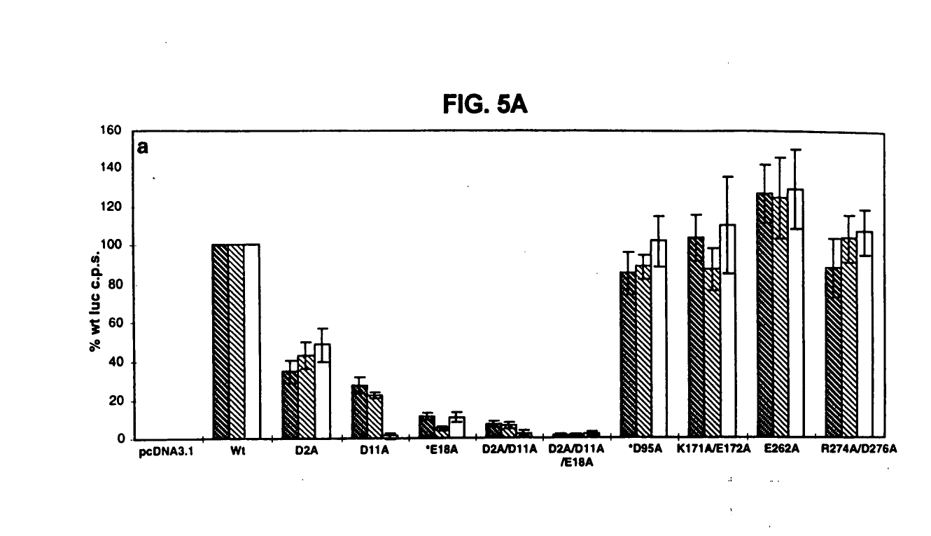

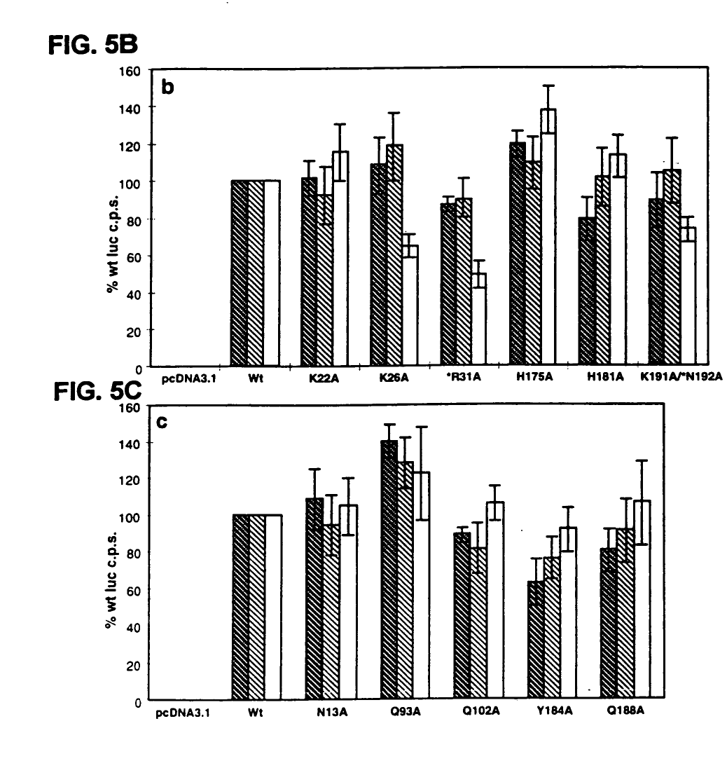

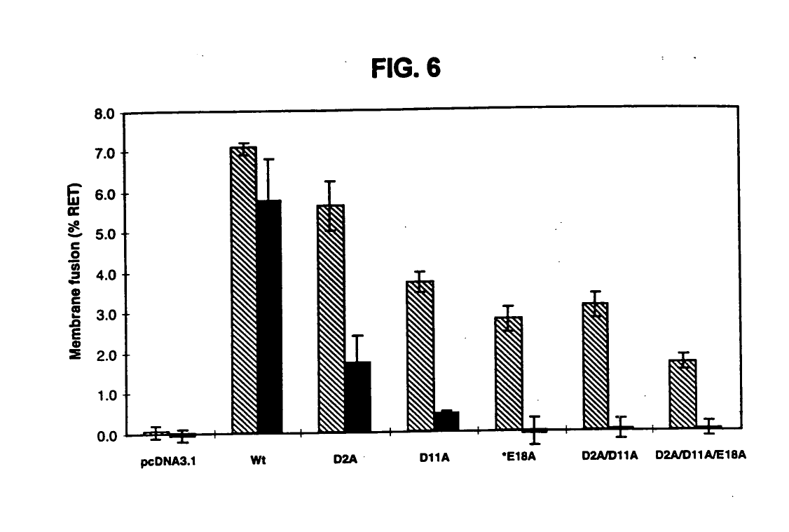

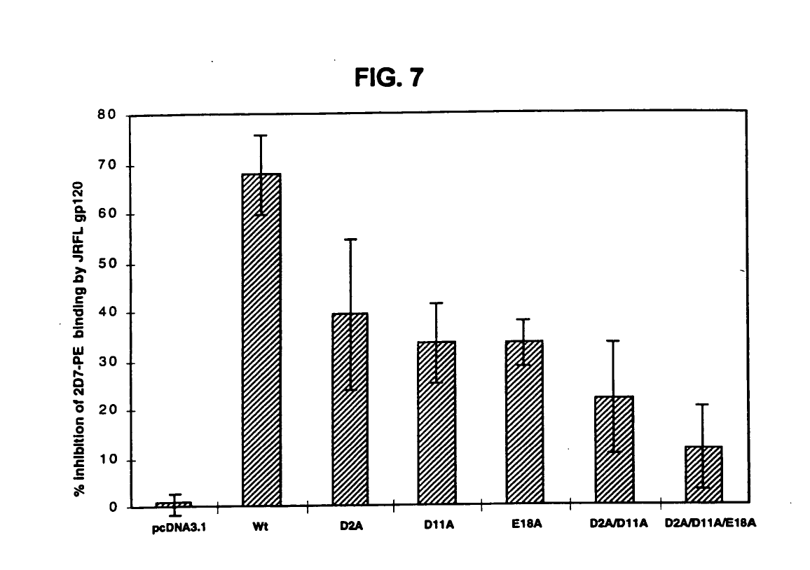

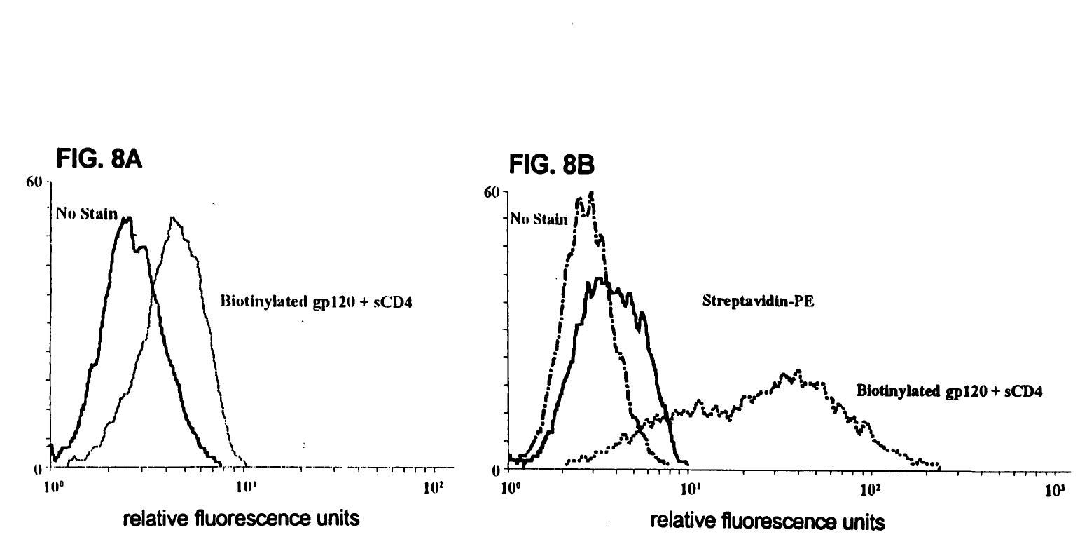

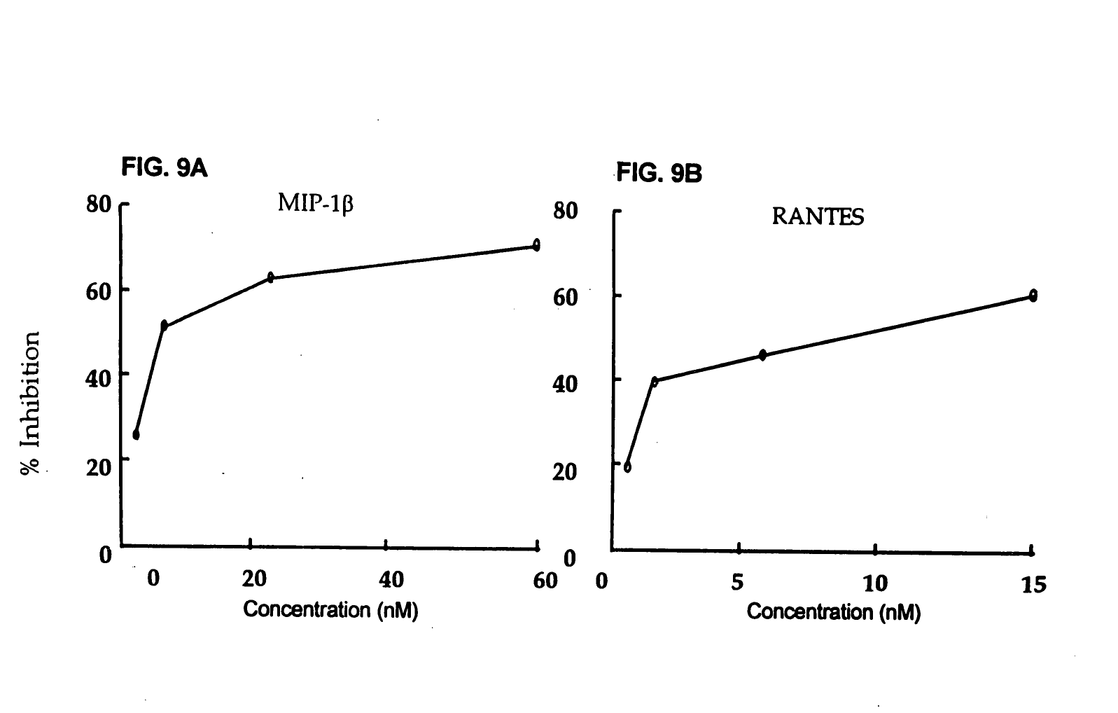

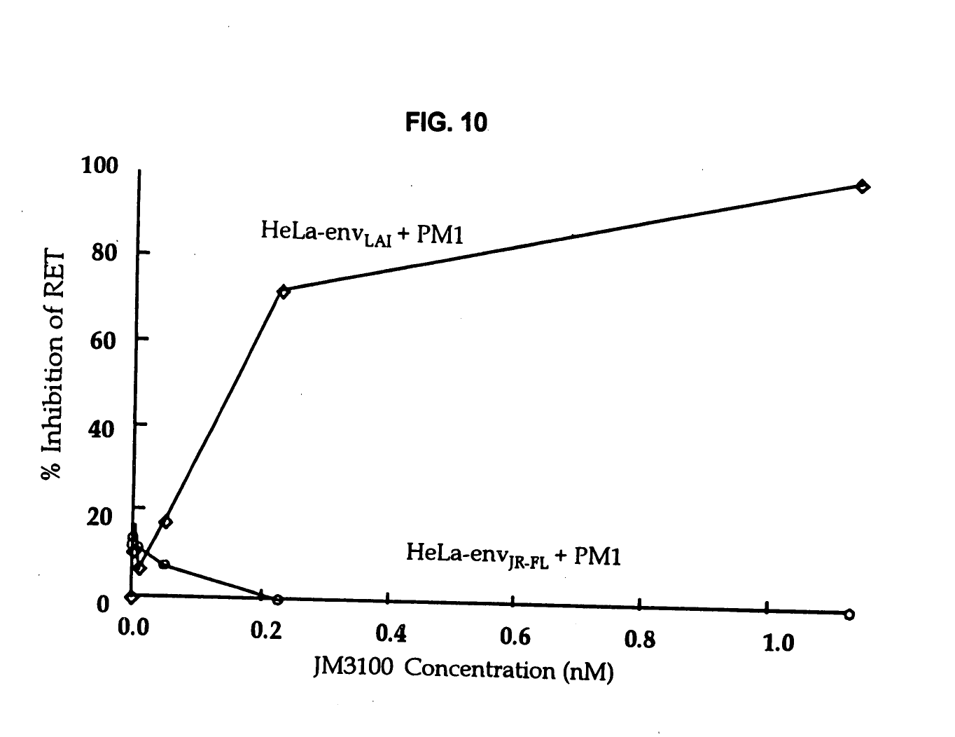

It was determined when β-chemokines inhibited HIV-1 replication by showing that complete inhibition of infection of PM1 cells required the continuous presence of β-chemokines for up to 5 h after addition of ADA or BaL env-complemented virus (FIG. 1a). Pre-treatment of the cells with β-chemokines for 2 h or 24 h prior to infection had no inhibitory effect if the cells were subsequently washed before virus addition. Furthermore, adding β-chemokines 2 h after virus only minimally affected virus entry (FIG. 1a). A PCR-based assay was next used to detect HIV-1 early DNA reverse transcripts in PM1 cells after 10 h of infection; reverse transcription of ADA, but not of NL4/3, could not be detected in the presence of MIP-1β and RANTES (FIG. 1b). Thus, inhibition by β-chemokines requires their presence during at least one of the early stages of HIV-1 replication: virus attachment, fusion and early reverse transcription.

These sites of action were discriminated, first by testing whether β-chemokines inhibited binding of JR-FL or BRU gp120 to soluble CD4, or of tetrameric CD4-IgG2 binding to HeLa-JR-FL cells expressing oligomeric envelope glycoproteins (17). No inhibition by any of the β-chemokines was found in either assay, whereas the OKT4a CD4-MAb was strongly inhibitory in both (data not shown). Thus, β-chemokines inhibit a step after CD4 binding, when conformational changes in the envelope glycoproteins lead to fusion of the viral and cellular membranes (18). Cell-cell membrane fusion is also induced by the gp120-CD4 interaction, and can be monitored directly by resonance energy transfer (RET) between fluorescent dyes incorporated into cell membranes (17). In the RET assay, OKT4a is completely inhibits membrane fusion of PM1 cells with HeLa cells expressing the envelope glycoproteins of either JR-FL (HeLa-JR-FL) or BRU (HeLa-BRU), confirming the specificity of the process (17). RANTES, MIP-1β (and to a lesser extent, MIP-1α) strongly inhibited membrane fusion of HeLa-JR-FL cells with PM1 cells, whereas fusion between PM1 cells and HeLa-BRU cells was insensitive to these β-chemokines (Table 2a).

TABLE 2
Effect of β-chemokines on HIV-1 envelope glycoprotein-mediated
membrane fusion measured using the RET assay

% Fusion

| | HeLa-JR-FL | HeLa-BRU |
|---|---|---|
| a) PM1 cells | | |
| no chemokines | 100 | 100 |
| +R/Mα/Mβ (80/400/100) | 1 | 95 |
| +RANTES (80) | 8 | 100 |
| +MIP-1α (400) | 39 | 100 |
| +MIP-1β (100) | 13 | 93 |
| +MCP-1 (100) | 99 | 98 |
| +MCP-2 (100) | 72 | 93 |
| +MCP-3 (100) | 98 | 99 |
| b) LW5 CD4⁺ cells | | |
| no chemokines | 100 | 100 |
| +R/Mα/Mβ(106/533/133) | 39 | 100 |
| +RANTES (106) | 65 | 95 |
| +MIP-1α (533) | 72 | 100 |
| +MIP-1β (133) | 44 | 92 |
| +OKT4A (3 ug/ml) | 0 | 0 |

Table 2 Legend:

CD4⁺ target cells (mitogen-activated CD4⁺ lymphocytes or PM1 cells) were labeled with octadecyl rhodamine (Molecular Probes, Eugene, Oreg.), and HeLa-JR-FL cells, HeLa-BRU cells (or control HeLa cells, not shown) were labeled with octadecyl fluorescein (Molecular Probes), overnight at 37° C. Equal numbers of labeled target cells and env-expressing cells were mixed in 96-well plates and β-chemokines (or CD4 MAb OKT4a) were added at the final concentrations (ng/ml) indicated in parentheses in the first column. Fluorescence emission values were determined 4 h after cell mixing (17). If cell fusion occurs, the dyes are closely associated in the conjoined membrane such that excitation of fluorescein at 450 nm results in resonance energy transfer (RET) and emission by rhodamine at 590 nm. Percentage fusion is defined as equal to 100×[(Exp RET−Min RET)/(Max RET−Min RET)], where Max RET=% RET obtained when HeLa-Env and CD4⁺ cells are mixed, Exp RET=% RET obtained when HeLa-Env and CD4⁺ cells are mixed in the presence of fusion-inhibitory compounds, and Min RET=% RET obtained when HeLa cells (lacking HIV-1 envelope glycoproteins) and CD4⁺ cells are mixed. The % RET value is defined by a calculation described elsewhere (17), and each is the mean of triplicate determinations. These values were, for HeLa-JR-FL and HeLa-BRU cells respectively: PM1 cells 11.5%, 10.5%; LW5 CD4⁺ cells, 6.0%, 10.5%; R/Mα/Mβ, RANTES+MIP-1α+MIP-1β.

Similar results were obtained with primary CD4⁺ T-cells from LW5 (Table 2b), although higher concentrations of β-chemokines were required to inhibit membrane fusion in the primary cells than in PM1 cells. Thus, the actions of the β-chemokines are not restricted to the PM1 cell line. The RET assay demonstrates that β-chemokines interfere with env-mediated membrane fusion.

The simplest explanation of these results is that the binding of certain β-chemokines to their receptor(s) prevents, directly or otherwise, the fusion of HIV-1 with CD4⁺ T-cells. It has been known for a decade that HIV-1 requires a second receptor for entry into CD4⁺ cells (19-21). This function is supplied, for TCLA strains, by fusin (9). Several receptors for MIP-1α, MIP-1β and RANTES have been identified (6, 7), and β-chemokines exhibit considerable cross-reactivity in receptor usage (4-8). However, C-C CKR-1 and, especially, C-C CKR-5 were identified as the most likely candidates, based on tissue expression patterns and their abilities to bind MIP-1α, MIP-1β and RANTES (4, 7, 8, 15, 22). C-C CKR-1, C-C CKR-5 and LESTR are each expressed at the mRNA level in PM1 cells and primary macrophages (data not shown). These and other β-chemokine receptors were therefore PCR-amplified, cloned and expressed.

The expression of C-C CKR-5 in HeLa-CD4 (human), COS-CD4 (simian) and 3T3-CD4 (murine) cells rendered each of them readily infectible by the primary, NSI strains ADA and BaL in the env-complementation assay of HIV-1 entry (Table 3).

TABLE 3
C—C CKR-5 expression permits infection of CD4-expressing
cells by primary, NSI HIV-1 strains

| | | | | | | | | | R/Mα/Mβ |
|---|---|---|---|---|---|---|---|---|---|
| | | pcDNA3.1 | LESTR | CKR-1 | CKR-2a | CKR-3 | CKR-4 | CKR-5 | CKR-5 |
| COS-CD4 | ADA | 798 | 456 | 600 | 816 | 516 | 534 | 153000 | 3210 |
| | BaL | 660 | 378 | 600 | 636 | 516 | 618 | 58800 | 756 |
| | HxB2 | 5800 | 96700 | 5240 | 5070 | 5470 | 5620 | 4850 | 5000 |
| HeLa-CD4 | ADA | 678 | 558 | 4500 | 912 | 558 | 600 | 310000 | 6336 |
| | BaL | 630 | 738 | 1800 | 654 | 516 | 636 | 104000 | 750 |
| | HxB2 | 337000 | nd | nd | nd | nd | nd | nd | 356000 |
| 3T3-CD4 | ADA | 468 | 558 | 450 | 618 | 534 | 606 | 28400 | 1220 |
| | BaL | 606 | 738 | 660 | 738 | 534 | 558 | 11700 | 756 |
| | HxB2 | 456 | 24800 | 618 | 672 | 732 | 606 | 618 | 606 |

Table 3 Legend:

Chemokine receptor genes C-C CKR-1, C-C CKR-2a, C-C CKR-3, C-C CKR-4 and C-C CKR-5 have no introns (4-8, 15, 22) and were isolated by PCR performed directly on a human genomic DNA pool derived from the PBMC of seven healthy donors. Oligonucleotides overlapping the ATG and the stop codons and containing BamHI and Xhol restriction sites for directional cloning into the pcDNA3.1 expression vector (Invitrogen Inc.) were used. LESTR (also known as fusin or HUMSTR) (4, 9, 24) was cloned by PCR performed directly on cDNA derived from PM1 cells, using sequences derived from the NIH database. Listed below are the 5′ and 3′ primer pairs used in first (5-1 and 3-1) and second (5-2 and 3-2) round PCR amplification of the CKR genes directly from human genomic DNA, and of LESTR from PM1 cDNA. Only a single set of primers was used to amplify CKR-5.

LESTR: L/5-1=AAG CTT GGA GAA CCA GCG GTT ACC ATG GAG GGG ATC (SEQ ID NO: 9);

L/5-2=GTC TGA GTC TGA GTC AAG CTT GGA GAA CCA (SEQ ID NO: 10);

L/3-1=CTC GAG CAT CTG TGT TAG CTG GAG TGA AAA CTT GAA GAC TC (SEQ ID NO: 11);

L/3-2=GTC TGA GTC TGA GTC CTC GAG CAT CTG TGT (SEQ ID NO: 12);

CKR-1:C1/5-1=AAG CTT CAG AGA GAA GCC GGG ATG GAA ACT CC (SEQ ID NO: 13);

C1/5-2=GTC TGA GTC TGA GTC AAG CTT CAG AGA GAA (SEQ ID NO: 14);

C1/3-1=CTC GAG CTG AGT CAG AAC CCA GCA GAG AGT TC (SEQ ID NO: 15);

C1/3-2=GTC TGA GTC TGA GTC CTC GAG CTG AGT CAG (SEQ ID NO: 16);

CKR-2a:C2/5-1=AAG CTT CAG TAC ATC CAC AAC ATG CTG TCC AC (SEQ ID NO: 17);

C2/5-2=GTC TGA GTC TGA GTC AAG CTT CAG TAC ATC (SEQ ID NO: 18);

C2/3-1=CTC GAG CCT CGT TTT ATA AAC CAG CCG AGA C (SEQ ID NO: 19);

C2/3-2=GTC TGA GTC TGA GTC CTC GAG CCT CGT TTT (SEQ ID NO: 20);

CKR-3: C3/5-1=AAG CTT CAG GGA GAA GTG AAA TGA CAA CC (SEQ ID NO: 21);

C3/5-2=GTC TGA GTC TGA GTC AAG CTT CAG GGA GAA (SEQ ID NO: 22);

C3/3-1=CTC GAG CAG ACC TAA AAC ACA ATA GAG AGT TCC (SEQ ID NO: 23);

C3/3-2=GTC TGA GTC TGA GTC CTC GAG CAG ACC TAA (SEQ ID NO: 24);

CKR-4: C4/5-1=AAG CTT CTG TAG AGT TAA AAA ATG AAC CCC ACG G (SEQ ID NO: 25);

C4/5-2=GTC TGA GTC TGA GTC AAG CTT CTG TAG AGT (SEQ ID NO: 26);

C4/3-1=CTC GAG CCA TTT CAT TTT TCT ACA GGA CAG CAT C (SEQ ID NO: 27);

C4/3-2=GTC TGA GTC TGA GTC CTC GAG CCA TTT CAT (SEQ ID NO: 28);

CKR-5: C5/5-12=GTC TGA GTC TGA GTC AAG CTT AAC AAG ATG GAT TAT CAA (SEQ ID NO: 29);

C5/3-12=GTC TGA GTC TGA GTC CTC GAG TCC GTG TCA CAA GCC CAC (SEQ ID NO: 30).

The human CD4-expressing cell lines HeLa-CD4 (P42), 3T3-CD4 (sc6) and COS-CD4 (Z28T1) (23) were transfected with the different pcDNA3.1-CKR constructs by the calcium phosphate method, then infected 48 h later with different reporter viruses (200 ng of HIV-1 p24/10⁶ cells) in the presence or absence of β-chemokines (400 ng/ml each of RANTES, MIP-1α and MIP-1β). Luciferase activity in cell lysates was measured 48 h later (10, 11). β-Chemokine blocking data is only shown for C-C CKR-5, as infection mediated by the other C-C CKR genes was too weak for inhibition to be quantifiable. In PCR-based assays of HIV-1 entry, a low level of entry of NL4/3 and ADA into C-C CKR-1 expressing cells (data not shown) was consistently observed.

Neither LESTR nor C-C CKR-1, -2a, -3 or -4 could substitute for C-C CKR-5 in this assay. The expression of LESTR in COS-CD4 and 3T3-CD4 cells permitted HxB2 entry, and HxB2 readily entered untransfected (or control plasmid-transfected) HeLa-CD4 cells (Table 3). Entry of BAL and ADA into all three C-C CKR-5-expressing cell lines was almost completely inhibited by the combination of MIP-1α, MIP-1β and RANTES, whereas HxB2 entry into LESTR-expressing cells was insensitive to β chemokines (Table 3). These results suggest that C-C CKR-5 functions as a β-chemokine-sensitive second receptor for primary, NSI HIV-1 strains.

The second receptor function of C-C CKR-5 was confirmed in assays of env-mediated membrane fusion. When C-C CKR-5 was transiently expressed in COS and HeLa cell lines that permanently expressed human CD4, both cell lines fused strongly with HeLa cells expressing the JR-FL envelope glycoproteins, whereas no fusion occurred when control plasmids were used (data not shown). Expression of LESTR instead of C-C CKR-5 did not permit either COS-CD4 or HeLa-CD4 cells to fuse with HeLa-JR-FL cells, but did allow fusion between COS-CD4 cells and HeLa-BRU cells (data not shown).

The fusion capacity of β-chemokine receptors was also tested in the RET assay. The expression of C-C CKR-5, but not of C-C CKR-1, -2a, -3 or -4, permitted strong fusion between HeLa-CD4 cells and HeLa-JR-FL cells. The extent of fusion between HeLa-JR-FL cells and C-C CKR-5-expressing HeLa-CD4 cells was greater than the constitutive level of fusion between HeLa-BRU cells and HeLa-CD4 cells (FIG. 2). The fusion-conferring function of C-C CKR-5 for primary, NSI HIV-1 strains has therefore been confirmed in two independent fusion assays.

Experimental Discussion

Together, the above results establish that MlP-1α, MIP-1β and RANTES inhibit HIV-1 infection at the entry stage, by interfering with the virus-cell fusion reaction subsequent to CD4 binding. It was also shown that C-C CKR-5 can serve as a second receptor for entry of primary NSI strains of HIV-1 into CD4+ T-cells, and that the interaction of β-chemokines with C-C CKR-5 inhibits the HIV-1 fusion reaction.

### REFERENCES OF THE PRECEDING SECTIONS

1. Levy, J. A., Mackewicz, C. E. & Barker, E. Immunol. Today 17, 217-224 (1996).
2. Cocchi, F. et al. Science 270, 1811-1815 (1995).
3. Paxton, W. A. et al. Nat. Med. 2, 412-417 (1996).
4. Neote, K., DiGregorio, D., Mak, J. Y., Horuk, R., & Schall, T. J. Cell 72, 415-425 (1993).
5. Gao, J.-L. et al. J. Exp. Med. 177, 1421-1427 (1993).
6. Bacon, K. B., Premack, B. A., Gardner, P. & Schall, T. J. Science 269, 1727-1729 (1995).
7. Raport, C. J. et al. J. Leukoc. Biol. 59, 18-23 (1996).
8. Wells, T. N. C. et al. J. Leukoc. Biol. 59, 53-60 (1996).
9. Feng, Y., Broder, C. C., Kennedy, P. E. & Berger, E. A. Science 272, 872-877 (1996).
10. Chen, B. K., Saksela, K., Andino, R. & Baltimore, D. J. Virol. 68, 654-660 (1994).
11. Connor, R. I., Chen, B. K., Choe, S., & Landau, N. R. Virology 206, 935-944 (1995).
12. Lusso, P. et al. J. Virol. 69, 3712-3720 (1995).
13. Charo, l. F. et al. Proc. Natl. Acad. Sci. USA 91, 2752-2756 (1994).
14. Ben-Baruch, A. et al. J. Biol. Chem. 270, 22123-22128 (1995).
15. Combadiere, C et al. J. Biol. Chem. 270, 29671-29675 (1995).
16. Lip, J. P., D'Andrea, A. D., Lodish, H. F. & Baltimore, D. Nature 343, 762-764 (1990).
17. Litwin, V. et al. J. Virol. (submitted for publication).
18. Moore, J. P., Jameson, B. A., Weiss, R. A. & Sattentau, Q. J. in Viral Fusion Mechanisms (ed Bentz, J.) 233-289 (CRC Press Inc, Boca Raton, USA, 1993).
19. Maddon, P. J. et al. Cell 47, 333-348 (1986).
20. Ashorn, P. A., Berger, E. A. & Moss, B. J. Virol. 64, 2149-2156 (1990).
21. Clapham, P. R., Blanc, D. & Weiss, R. A. Virology 181, 703-715 (1991).
22. Samson, M., Labbe, O., Mollereau, C., Vassart, G. & Parmentier, M. Biochemistry 11, 3362-3367 (1996).
23. Dragic, T., Charneau, P., Clavel, F. & Alizon, M. J. Virol. 66, 4794-4802 (1992)
24. Loetscher, M. et al. J. Biol. Chem. 269, 232-237 (1994).
25. Moore, J. P. & Ho, D. D. AIDS 9 (suppl A), S117-S136 (1995).
26. Trkola, A. & Moore, J. P. (unpublished data).
27. Chaudhuri, A., et al. 1994. J. Biol. Chem. 269, 7835-7838 (1994).
28. Neote, K., Mak, J. Y., Kolakowski Jr., L. F. & Schall, T. J. Blood 84, 44-52 (1994).
29. Dragic, T., Picard, L. & Alizon, M. J. Virol. 69, 1013-1018 (1995).
30. Puri, A., Morris, S. J., Jones, P., Ryan, M. & Blumenthal, R. Virology 219, 262-267 (1996).31
31. Choe, H., Farzan, F., Sun, Y., Sullivan, N., Rollins, B., Ponath, P. D., Wu, L., Mackay, C. R., LaRosa, G., Newman, W., Gerard, N., Gerard, C., and Sodroski, J. Cell 85, 1135-1148 (1996.
32. Doranz, B. J., Rucher, J., Yi, Y., Smyth, R. J., Samson, M., Peiper, S. c., Parmentier, M., Collman, R. G., and Doms, R. w. Cell 85, 1149-1158, (1996).
33. Wu, L., Paxton, W. A., Kassam, N., Ruffing, N., Rottman, J. B., Sullivan, N., Choe, H., Sodroski, J., Newman, W., Koup, R. A., and Mackay, C. R. J. Exp. Med. 185, Number 9, 1681-1691, (1997).
   Second Series of Experiments
   Experimental Methods

Sources of reagents. Recombinant human β-chemokines were purchased from R&D Systems (Minneapolis) and ¹²⁵I-MIP-1β (specific activity 2,200 Ci mmol⁻¹) was from Dupont-NEN. Anti-CD4 monoclonal antibodies were from Q. Sattentau (16), except for 5A8, from L. Burkly (Biogen) (27) and L120 (UK MRC AIDS Reagent Repository) (16). Soluble CD4 has been described (17). Monoclonal antibodies to gp120 were obtained from donors listed elsewhere (19, 20), except for 23A (gp120 C terminus, from J. Robinson), 447-D and 697-D (ref. 28; Cellular Products Inc.) And 83.1 (ref. 29; Repligen). Recombinant gp41 (IIIB) ectodomain was from Viral Therapeutics Inc., and V3 peptides were obtained either from Repligen or the UK MRC AIDS Reagent Repository. Recombinant MN gp120 (Genentech), SF-2 gp120 (Chiron) and CM243 gp120 were provided by the NIAID AIDS Reagents Repository, and W61D gp120 (SmithKline Beecham, Belgium) was from the UK MRC AIDS Reagent Repository.

Recombinant, monomeric JR-FL and LAI gp120s, both full-length and with the V3-loops deleted, were expressed using vectors developed at Progenics Pharmaceuticals that contain a dihydrofolate reductase expression cassette. The expression of the gene is under the control of the cytomegalovirus immediate-early promoter. For Δ-V3 gp120s, the V3 loops were excised by the splicing-by-overlap-extension technique, such that the cysteines defining the loop were retained and spanned by the peptide sequence TGAGH. All constructs were sequenced to verify that no mutations were introduced during the cloning manipulations. The proteins were expressed in stably transfected Chinese hamster ovary cells (DXB-11), selected in nucleoside-free medium and amplified using methotrexate, following previously described methods (17). The secreted proteins were purified to >95% homogeneity in a non-denaturing process comprising an ion exchange, Galanthus nivalus lectin affinity and gel filtration chromatography. The purified proteins bound sCD4 with nanomolar affinity (18).

For expression of SF162 gp160, a 3.5-kb EcoR1-BamH1 fragment containing the env gene was excised from the SV40-based vector pSM and subcloned into the R1/Bg1lll sites of the β-actin-based expression vector pCAGGS. For expression of SF170 gp160, a 3.8-kb fragment containing the env gene was excised from the pBSKS⁺ plasmid, blunted by treatment with T4 DNA polymerase, and subcloned into the RV/Xhol sites of pCAGGS. The expression plasmids were transfected into 293 T cells by calcium phosphate co-precipitation. Soluble gp120 in the culture supernatant was collected after three days, filtered through 0.2-μm filters and concentrated over an Amicon 1000 membrane.

The preparation of soluble, oligomeric forms of the JR-FL and 94RW020 envelope glycoproteins (and also monomeric gp120 from 92TH014) was as follows. The JR-FL env gene was provided by I. Chen (UCLA) and the env genes of 92TH014 and 92RW020 were obtained from the NIAID AIDS Reagent Repository (30). Soluble expression plasmids encoding gp120 and the gp41 ectodomain of JR-FL and 92RW020, gp120 only of 92TH014, were constructed as described (30), and transfected into Chinese hamster ovary cells by the calcium phosphate method. The cleavage sites between the JR-FL and 92TH014 gp120 and gp41 moieities were retained, and proteins secreted as oligomers (J. A., J. M. B. and J. P. M., unpublished data). Envelope glycoproteins were partially purified from culture supernatants by immobilized metal-affinity chromatography. A control preparation, 93MW959(c), containing a gp120/gp41 molecule incompetent at CD4 binding, by virtue of a single point mutation at residue 457, did not compete with ¹²⁵I-MIP-1β. The monomeric gp120 or oligomeric gp120/gp41 concentrations in unpurified culture supernatants were estimated by denaturing the proteins (19), then dot-blotting onto nitrocellulose membranes and detecting the gp120 with a cocktail of murine monoclonal antibodies to continuous epitopes (19), followed by an anti-mouse IgG-HRP conjugate and the ECL chemiluminescence system (Amersham). Purified, monomeric JR-FL gp120 was used as a concentration standard (17). The concentration of oligomeric gp120/gp41 complexes was defined as the total concentration of monomeric gp120 subunits in the preparation. High-affinity CD4 binding of the gp120s was confirmed by enzyme-linked immunosorbent assay (ELISA)(19).

Cells and cell lines. PBMCs were isolated from blood donors by Ficoll-Hypaque centrifugation, and stimulated for 2-3 days with phytohaemagglutnin (5 μg ml⁻¹) and IL-2 (100 U ml⁻¹) (Roche). CD4⁺ T cells were purified from the activated PBMCs by positive selection using anti-CD4 immunomagnetic beads (Dynal Inc.). The purified lymphocytes were cultured for at least 3 days at 2×10⁶/ml in medium containing IL-2 (200 U ml⁻¹) before being used in the ¹²⁵I-MIP-1β binding assay. The cells were screened for CCR-5-defective alleles (14), and only cells from wild-type donors were used (except when specified). 293 cells were transfected with pcDNA3.1-ckr-5 (ref. 1) using the calcium phosphate method, and resistant clones were selected in culture medium containing 1 μg ml⁻¹ neomycin (G418; Sigma). Resistant cells were subcloned and tested for CCR-5 expression in a binding assay using ¹²⁵I-MIP-1β.

MIP-1β binding assay and gp120 competition. Purified CD4⁺ T cells were washed twice in ice-cold binding buffer (RPMI 1640 medium containing 1% BSA, 25 mM HEPES, 0.05% sodium azide). Duplicate samples (2×10⁶ cells in 200 μl) were incubated with 0.1 nM ¹²⁵I-MIP-1β (2,200 Ci mmol⁻¹; 0.25 μCml⁻¹) for 2 h on ice. Unlabelled ligand or gp120 (mixed with monoclonal antibodies when appropriate) was added to the cells immediately before radiolabelled ligand was added. Anti-CD4 monoclonal antibodies were added to the cells simultaneously with gp120. These cells were then separated from unbound ligand by centrifugation (60 s, 14,000 g) through oil (80% silicone oil, Aldrich; 20% mineral oil, Sigma), and the radioactivity in the cell pellet was determined by gamma counting. Specific binding of ¹²⁵I-MIP-1β was estimated by including a 100-fold excess of unlabelled MIP-1β. Each experiment was repeated at least twice using cells from different donors. For experiments with 293-CCR-5 cells, the cells were detached with 1 mM EDTA then washed twice with binding buffer. Samples (5×10⁵ cells) were incubated with 0.5 nM ¹²⁵I-MIP-1β, then processed as above. When the ¹²⁵I-MIP-1β concentration was reduced to 0.1 nM, no specific binding was detected.

### SUMMARY

The β-chemokine receptor CCR-5 is an essential co-factor for fusion of HIV-1 strains of the non-syncytium-inducing (NSI) phenotype with CD4⁺ T-cells (1-5). The primary binding site for human immunodeficiency virus (HIV)-1 is the CD4 molecule, and the interaction is mediated by the viral surface glycoprotein gp120 (6, 7). The mechanism of CCR-5 function during HIV-1 entry has not been defined, but we have shown previously that its β-chemokine ligands prevent HIV-1 from fusing with the cell (1). We therefore investigated whether CCR-5 acts as a second binding site for HIV-1 simultaneously with or subsequent to the interaction between gp120 and CD4. We used a competition assay based on gp120 inhibition of the binding of the CCR-5 ligand, macrophage inflammatory protein (MIP)-1β, to its receptor on activated CD4⁺ T cells or CCR-5 positive CD4⁻ cells. We conclude that CD4 binding, although not absolutely necessary for the gp120-CCR-5 interaction, greatly increases its efficiency. Neutralizing monoclonal antibodies against several sites on gp120, including the V3 loop and CD4-induced epitopes, inhibited the interaction of gp120 with CCR-5, without affecting gp120-CD4 binding. Interference with HIV-1 binding to one or both of its receptors (CD4 and CCR-5) may be an important mechanism of virus neutralization.

MIP-1β is the most specific ligand for CCR-5 (8-10) because MIP-1α and RANTES also bind with high affinity to other members of the β-chemokine receptor family on lymphoid cells (8-11). We therefore used MIP-10 as the CCR-5 ligand in the competition assays. In common with other members of this receptor family (12), CCR-5 is a mitogen-response gene. Its expression in quiescent, purified CD4⁺ T-cells is usually minimal, but 3 days after activation of the cells by phytohaemagglutinin and interleukin (IL)-2, we observed strong increases in CCR-5 messenger RNA and ¹²⁵I-labelled-MIP-1β binding (data not shown). As specificity controls, we used CD4⁺ T cells from individuals homozygous for defective CCR-5 alleles (13, 14). The amount of specific (that is, cold MIP-1β-competed) ¹²⁵I-MIP-1β (0.1 nM) binding to cells from three such individuals was 92±12 c.p.m. per 2×10⁶ cells (mean±s.d.). In contrast, mean binding to cells from 21 control individuals was 1,044±1,073 c.p.m. per 2×10⁶ cells (range, 222-4,846 c.p.m.). Most of the ¹²⁵I-MIP-1β reactivity with activated CD4⁺ T cells therefore derives from binding to CCR-5.

When recombinant, monomeric gp120s were added with ¹²⁵I-MIP-1β to activated CD4⁺ T cells, we found that gp120 from the NSI strain JR-FL [which used CCR-5 for entry (1)] strongly inhibited MIP-10 binding (FIG. 3a: Table 4).

TABLE 4
Effect of recombinant gp120 on MIP-Iβ binding

| gp120 (μg ml⁻¹) | | | | | | |
|---|---|---|---|---|---|---|
| 0.1 | 0.2 | .05 | 5 | 20 | 50 | V3 sequence |

| | | | | | | | |
|---|---|---|---|---|---|---|---|
| NSI gp120 | | | | | | | |
| JR-FL (B) | 44 ± 9 | 40 ± 23 | 58 ± 26 | 67 ± 9 | 91 ± 3 | 95 ± 6 | CTRPNNNTRKSIHIGPGRAFYTTGEIIGDIRQAHC |
| JR-FL (B)* | 46 ± 28 | 56 ± 3 | 84 ± 11 | | | | |
| SF 162 (B) | 81 ± 8 | 113 ± 41 | | | | | CTRPNNNTRKSITIGPGRAFYATGDIIGDIRQAHC |
| W61D (B) | 39 ± 14 | 53 ± 17 | 65 ± 23 | | 86 ± 12 | | CTRPNNNTRKGIHIGPGRAFYAARKIIGDIRQAHC |
| 92TH014 (B) | 6 ± 1 | 57 ± 16 | 65 ± 5 | | | | CTRPNNNTRKSIHLGPGRAWYTTGQIIGDIRQAHC |
| SF 170 (A) | 87 ± 45 | 140 ± 49 | | | | | CTRPNNNTRKSVRIGPGQAFYATGDIIGDIRQAYC |
| 92RW020 (A)* | 21 ± 39 | 49 ± 22 | 74 ± 36 | | | | CTRPNNNTRKGVRIGPGQAFYTGQIIGDIRQAHC |
| CM243 (E) | 17 ± 14 | 40 ± 11 | 56 ± 38 | | 76 ± 24 | | CTRPSNNTRPSITVGPGQVFYRTGDIIGDIRRAYC |
| TCLA gp120 | | | | | | | |
| LAI (B) | | | −1 ± 3 | −5 ± 36 | −18 ± 39 | −6 ± 9 | CTRPNNNTRKSIRIQRGPGRAFVTIGKIGNMRQAHC |
| MN (B) | | | −20 ± 14 | −23 ± 4 | 1 ± 0 | 0 ± 6 | CTRPNYNKKRIHIGPGRAFYTTKNIIGTIRQAHC |
| SF-2 (B) | | | −1 ± 4 | 3 ± 3 | 8 ± 26 | 21 ± 3 | CTRPNNNTRKSIYIGPGRAFHTTGRIGDIRKAHC |
| gp41 | | | | | | | |
| IIIB (B) | | | | 24 ± 5 | | | |

Table 4 Legend:

Recombinant proteins were titrated in the presence of 0.1 nM ¹²⁵I-MIP-1β and added to activated CD4⁺ T cells. Percentage inhibitions of ¹²⁵I-MIP-1 binding at each gp120 concentration are shown, and are the means±s.d. of 2-4 independent experiments. No value indicates that the gp120 molecule was not tested at that concentration (several molecules were not available at concentrations >1 μg ml⁻¹). * An oligomeric gp120/gp41 complex.

Half-maximal inhibition occurred in the range 0.1-1.0 μg ml⁻¹ (0.8-8 nM) gp120, which is similar to the association constant for the gp120-CD4 interaction (7). In contrast, gp120 from the T-cell-line adapted (TCLA) SI strain LAI was ineffective (FIG. 3a; Table 4). This virus uses fusin (CXCR-4), but not CCR-5, for entry (1-5). Mutants of JR-FL and LAI gp120s which lack the V3 loop (Δ-V3 gp120), but bind CD4 with high affinity, did not block MIP-1β binding (FIG. 3a). However, peptides (15-residue if not specified) from the V3 loops of the following strains were also inactive: JR-FL (32-residue), RA, VS, Case-B (each NSI); HxB2, MN, SF-2 (each TCLA) (peptides were added at 1 μg ml⁻¹, the approximate molar equivalent of 60 μg ml⁻¹ gp120). An oligomeric complex of JR-FL gp120 noncovalently associated with the ectodomain of gp41 was an effective inhibitor of MIP-1βbinding, but a recombinant molecule comprising only the gp41 ectodomain was not (Table 4), although the latter molecule may not fold into a native structure (15).

HIV-1 strains from genetic subtypes A, B, C and E can use CCR-5 for entry (3), and we have found that MIP-1β inhibits the replication of most primary, NSI HIV-1 strains from subtypes A to E. This breadth of reactivity of HIV-1 with CCR-5 extend to the ¹²⁵I-MIP-1β competition assay. Recombinant gp120s from the following NSI primary strains were competitive, with half-maximal inhibition of MIP-1β binding occurring at concentrations around 0.1-0.5 μg ml⁻¹: JR-FL (subtype B), SF162 (B), W61D (B), 92TH014 (B), SF170 (A), 92RW020 (A) and CM243 (E) (Table 4). In contrast, no competition was observed with gp120s from the TCLA subtype B strains LAI, MN and SF-2 (Table 4), although each could bind CD4 with high affinity (not shown). Thus the phenotype of the virus from which a gp120 molecule is derived is more important than the viral genotype in determining interactions with CCR-5.

We assessed the role of CD4 in the competition between NSI gp120 and MIP-1β by using antagonists of the gp120-CD4 interaction. The monoclonal antibodies Q4120 and L77, which react with domain 1 of CD4 to inhibit gp120 binding (16), and soluble CD4 (sCD4), which reacts with gp120 to inhibit CD4 binding (17), both reversed the inhibition by JR-FL gp120 of MIP-1β binding to CCR-5 (FIG. 3b; Table 5).

TABLE 5
Monoclonal antibody inhibition of the gp120 interaction with CCR-5

| | | Monoclonal | | |
|---|---|---|---|---|
| | Epitope | antibody | Inhibition (%) | ±s.d. |
| CD4 antibodies | CD4-D1 | Q4120 | 83 | 27 |
| | | L77 | 77 | 12 |
| | CD4-D2 | 5A8 | 3 | 15 |
| | CD4-D3 | Q425 | 15 | 23 |
| | CD4-D4 | L120 | −17 | 23 |
| anti-gp120 antibodies | C5 | D7324 | −13 | 1 |
| non-neutralizing face | | 23A | −11 | 14 |
| | C1 (D) | CRA-1 | −2 | 18 |
| | | 522-149 | 9 | 1 |
| | C1 (L) | 133/192 | 8 | 7 |
| | C1-C4 | A32 | 51 | 11 |
| | | 211c | 5 | 11 |
| | C1-C5 | C11 | 3 | 21 |
| anti-gp120 antibodies | CD4bs | sCD4* | 91 | 18 |
| neutralizing face | CD4bs | 15e† | 65 | 18 |
| | | IgG1b12* | 125 | 40 |
| | C4 (L) | G3 508† | 57 | 1 |
| | C4-V3 (D) | G3 42† | 13 | 10 |
| | CD4i | 48d* | 54 | 33 |
| | | 17b* | 79 | 41 |
| | V2 | 697-D† | 3 | 37 |
| | | SC258 | −3 | 8 |
| | V3 | 447-D* | 109 | 2 |
| | | 19b† | 88 | 12 |
| | | 83.1† | 140 | 48 |
| | | 2G12* | 38 | 4 |

Table 5 Legend:

JR-FL gp120 (2 ug ml⁻¹) inhibition of ¹²⁵I-MIP-1β binding to activated CD4⁺ T cells was tested in the presence or absence of sCD4 (50 μg ml⁻¹) or monoclonal antibodies to CD4 (50 μg ml⁻¹) or antibodies to gp120 (20 μg ml⁻¹). Mean percentage reversals of the competitive effect of gp120 in the presence of each antibody (±s.d; n=2-4 independent experiments) are shown. The level of specific ¹²⁵I-MIP-1β binding (c.p.m.) Recorded in the presence of gp120 but the absence of antibody was set at 0%, and the level recorded in the absence of both gp120 and antibody was set at 100%. A negative percentage reversal indicates that the competitive effect of gp120 on ¹²⁵I-MIP-1β binding was increased in the presence of the antibody. Also listed are the approximate positions of the antibody epitopes on gp120, as defined (19, 20). References to the origin of the antibodies are described elsewhere (19, 20) or listed in the Methods section.

Monoclonal antibodies to other domains of CD4 [which do not block gp120-CD4 binding (16)] were ineffective (Table 5) and, in the absence of gp120, sCD4 (50 μg ml⁻¹) caused no inhibition of MIP-1β binding (data not shown). An interaction with cell-surface CD4 is therefore important for gp120 to interact efficiently with CCR-5 and block MIP-1β binding. To determine whether CD4 was an absolute requirement, we prepared a stable human CD4⁻ CCR-5⁺ 293 cell line. These cells bind ¹²⁵I-MIP-1β (specific binding up to 2,500 c.p.m. per 5×10⁵ cells), whereas untransfected 293 cells do not (specific binding <50 c.p.m.). The binding of ¹²⁵I-MIP-1β to the CD4-CCR-5⁺ 293 cells was sporadically inhibited by JR-FL gp120, but only at the highest gp120 concentrations tested (50-100 μg ml⁻¹). The strongest competition observed on these cells was 73% inhibition of MIP-1β binding by 50 μg ml⁻¹ of JR-FL gp120 (comparable inhibition was found in two other experiments), but competition was often not detected at all, and we never observed inhibition of MIP-1 binding at lower concentrations of gp120. The addition of excess sCD4 to the CD4⁻ CCR-5⁺ cells neither reduced nor increased the inhibitory effect of JR-FL gp120 (data not shown).

The interaction between JR-FL gp120 and CCR-5 requires at least 100-fold higher gp120 concentrations on CD4⁻ cells than on CD4⁺ cells. We suggest this is because binding of gp120 to CD4 on the cell surface increases the probability of a gp120-CCR-5 interaction; either a gp120-CD4-CCR-5 ternary complex forms, or there are sequential interactions of gp120 with CD4, then CCR-5. One possibility is that the high-affinity association of gp120 with CD4 increases the probability of a lower-affinity interaction of gp120 with CCR-5 (a proximity effect). This is consistent with the finding that sCD4 does not substitute for cell-surface CD4, at least with JR-FL gp120. Alternatively, binding to CD4 may be necessary to (better) expose a CCR-5 binding site on gp120. This may be especially important in the context of virions, where some regions of the oligomeric envelope glycoproteins (including the V3 loop) that are accessible on monomeric gp120 are not optimally exposed before CD4 binding (18).

To gain further sight into how the gp120-CD4 complex interacts with CCR-5 on activated CD4⁺ T cells, we used a panel of HIV-1 neutralizing and non-neutralizing anit-gp120 monoclonal antibodies (19, 20) having confirmed that each could bind to JR-FL gp120. The antibodies were tested for reversal of the competitive effect of gp120 on MIP-1β binding site (Table 5). As with sCD4, the antibodies to conformational (15e and IgG1b12) or linear (G3-508) epitopes overlapping the CD4-ginding site (20) prevented JR-FL gp120 from competing with MIP-1β. However, several antibodies that do not affect the binding of monomeric gp120 to CD4 (20) also inhibited the gp120-CCR-5 interaction (Table 5). These included three (447-D, 10b and 83.1) to the V3 loop; one (2G12) to a conserved epitope in the C3-V4 region; two (48d and 17b) to a conserved, CD4-induced epitope. All of these except A32 map to what we have defined as the gp120-neutralizing face (20). Eight other monoclonal antibodies that did not prevent JR-FL gp120 from blocking MIP-1β binding (Table 2) cluster on what we have defined as the gp120-non-neutralizing face (20): their epitopes are accessible on monomeric gp120, but in the context of the oligomer they are occluded either by other gp120 subunits or by gp41 molecules (19, 20). These ineffective antibodies include 2/11c to an epitope overlapping that of A32; for this reason, and because A32 neutralizes no HIV-1 strains strongly, the significance of the partial inhibitory action of A32 on the gp120-CCR-5 interaction is uncertain. Two monoclonal antibodies (697-D and SC259) to the V2 loop were also ineffective; although the V2 loop structure is modelled as being on (or above) the gp120 neutralizing face (20), these two antibodies are unable to neutralize HIV-1_{JR-FL}. The monoclonal antibodies 2G12, 17b, 447-D, 48d, IgG1b12, G3-508 and 697-D were also tested against the oligomeric JR-FL gp120/gp41 protein, and all except 697-D inhibited the interaction of this protein with CCR-5 (not shown).

Most of these antibodies to the neutralizing face of gp120 therefore either prevented gp120 from binding to CD4 or interfered with subsequent interactions with the CCR-5 second receptor. Not every antibody to this face of gp120 actually neutralizes HIV-1_{JR-FL}, as primary viruses resist neutralization, and studies with recombinant proteins can only predict neutralization efficiencies imprecisly (18). However, our findings may have implications for understanding how HIV-1 is neutralized by antibodies; blockade of the primary or secondary receptor interactions of the virus may be particularly important.

The simplest interpretation of the inability of Δ-V3 JR-FL gp120 to block MIP-1β binding (FIG. 3a) is that the CCR-5 binding site is contained within the V3 loop. This would be consistent with the many observations that the V3 loop contains important determinants of HIV-1 phenotype and tropism (18, 21), and can influence second-receptor usage (3). We believe, however, that the CCR-5 binding site is not limited to the V3 loop. The V3 sequences of gp120s of subtypes A, B and E that interact with CCR-5 are rather variable (Table 4). Furthermore, some simian immunodeficiency virus (SIV) strains can also use human CCR-5 as a second receptor (Z. W. Chen and P. Marx, personal communication), but the V3 regions of HIV-1 and SIV have almost no primary sequence homology. Can all these sequences each form a binding site for the same, conserved cellular protein, when similar V3 sequences from TCLA HIV-1 strains cannot (Table 4)? Twin-site models of the interaction of ligands with chemokine receptors (8) leave open the possibility that a relatively conserved section of the V3 loop could be one component of a multi-point binding site for CCR-5 on gp120. However, we suggest that the CCR-5 binding site must include a region of gp120 that is strongly conserved across the primate immunodeficiency viruses, not just across the HIV-1 genetic subtypes. Whether this is also the case for HIV-1 interactions with CXCR-4 remains to be determined.

The structure of the V3 loop may influence the nature of a complex binding site for CCR-5 on gp120. A region of gp120 overlapped by (but not necessarily identical to) the CD4-induced epitopes of monoclonal antibodies 48d and 17b is a good candidate for such a site. These antibodies recognize similar conformationally sensitive structures that are probably located around the bases of the V1, V2 and V3 loops (22, 23). Deletion of the V3 loop from both HxBc2 gp120 and JR-FL gp120 destroys the 48d and 17b epitopes (22, 23) (unpublished data), which may be relevant to the inability of the Δ-V3 JR-FL gp120 to interact with CCR-5 (FIG. 5A), and single amino-acid changes in the V3 and C4 regions of HIV-1_{LAI} also have major effects on the structure of these epitopes (24).

Further studies will be required to refine our understanding of the CCR-5 binding site. The efficiency with which β-chemokines inhibit the replication of NSI primary isolate in peripheral blood mononuclear cells (PMBCs) is dependent on strain but not subtype (unpublished data), suggesting, perhaps, that the degree of overlap between the gp120s and β-chemokine binding sites on CCR-5 varies between gp120s. If so, the CCR-5 binding site on gp120 might be more flexible than the CD4 binding site. Finally, although sCD4 inhibited the interaction between JR-FL gp120 and CCR-5 on CD4⁺ cells, for some strains of HIV-1 and (especially) HIV-2 and SIV, sCD4 might enhance the efficiency of second-receptor interactions, and thereby facilitate the entry of these primate immunodeficiency viruses into CD4 or CD4⁺ cells (25, 26).

### REFERENCE OF THE SECOND SERIES OF EXPERIMENT

1. Dragic, T. et al. Nature 381, 667-673 (1996).
2. Deng, H. K. et al. Nature 381, 661-666 (1996).
3. Choe, H. et al. Cell 86, 1135-1148 (1996).
4. Doranz, B. J. et al. Cell 86, 1149-1159 (1996).
5. Alkhatib, G. et al. Science 272, 1955-1958 (1996).
6. Maddon, P. J. et al. Cell 47, 333-348 (1986).
7. Lasky, L. A. et al. Cell 50, 975-985 (1987).
8. Wells, T. N. C. et al. J. Leuk. Biol. 59, 53-60 (1996).
9. Samson, M., Labbe, O., Mollereau, C., Vassart, G. & Parmentier, M. Biochemistry 11, 3362-3367 (1996).
10. Raport, C. J., Gosling, J., Schweickart, V. L., Gray, P. W. & Charo, I. F. J. Biol. Chem. 271, 17161-17166 (1996).
11. Neote, K., DiGregorio, D., Mak, J. Y., Horuk, R. & Schall, T. J. Cell 72, 415-425 (1993).
12. Loetscher, P., Seitz, M., Baggiolini, M. & Moser, B. J. Exp. Med. 184, 569-578 (1996).
13. Paxton, W. A. et al. Nature Med. 2, 412-417 (1996).
14. Liu, R. et al. Cell 86, 367-378 (1996).
15. Weissenhorn, W. et al. EMBO J. 15, 1507-1514 (1996).
16. Healey, D. et al. J. Exp. Med. 172, 1233-1242 (1990).
17. Allaway, G. P. et al. AIDS Res. Hum. Retrovirus 11, 533-540 (1995).
18. Moore, J. P. & Ho, D. D. AIDS 9 (suppl. A), S117-S136 (1995).
19. Moore, J. P. & Sattentau, Q. J., Wyatt, R. & Sodroski, J. J. Virol. 68, 469-484 (1994).
20. Moore, J. P. & Sodroski, J. J. Virol. 70, 1863-1872 (1996).
21. Cheng-Mayer, C. AIDS 4 (suppl. 1), S49-S56 (1990).
22. Thali, M. et al. J. Virol. 67, 3978-3988 (1993).
23. Wyatt, R. et al. J. Virol. 69, 5723-5733 (1995).
24. Moore, J. P., Yoshiyama, H., Ho, D. D., Robinson, J. E. & Sodroski, J. AIDS Res. Hum. Retroviruses 9, 1179-1187 (1993).
25. Sullivan, N., Sun, Y., Li, J., Hoffmann, W. & Sodroski, J. J. Virol. 69, 4413-4422 (1995).
26. Allan, J. S., Strauss, J. & Buck, D. W. Science 247, 1084-1088 (1990).
27. Burkly, L. C. et al. J. Immunol. 149, 1779-1787 (1992).
28. Gorny, M. K. et al. J. Virol. 68, 8312-8320 (1994).
29. White-Scharf, M. E. et al. Virology 192, 197-208 (1993).
30. Lu, S. et al. J. Virol. 70, 3978-3991 (1996).
   Third Series of Experiments
   Summary

The CC-chemokine receptor CCR5 is required for the efficient fusion of M-tropic HIV-1 strains with the plasma membrane of CD4+ cells (1-5), and interacts directly with the viral surface glycoprotein gp120 (6, 7). Although receptor chimera studies have provided useful information (8-10), the domains of CCR5 that function for HIV-1 entry, including the site of gp120 interaction, have not been unambiguously identified. Here, we use site-directed, alanine-scanning mutagenesis of CCR5 to show that only substitutions of the negatively charged residues Asp-2 (D2), Asp-11 (D11) and Glu-18 (E18), singly or in combination, impair or abolish CCR5-mediated HIV-1 entry for the ADA and JR/FL M-tropic strains and the DH123 dual-tropic strain. These mutations also impair env-mediated membrane fusion and the gp120-CCR5 interaction. Of these three residues, only D11 is necessary for CC-chemokine-mediated inhibition of HIV-1 entry, which is, however, also dependent on other extracellular CCR5 residues. Thus, the gp120 and CC-chemokine binding sites on CCR5 are only partially overlapping, and the former requires negatively charged residues in the N-terminal CCR5 domain.

Result

To identify regions of CCR5 involved in gp120 binding and HIV-1 entry, we performed alanine-scanning mutagenesis of negatively (D, E) or positively (K, R, H) charged residues in the N-terminus (Nt) and three extracellular loops (ECL 1-3), on the grounds that charged residues have been previously implicated in the interactions of CC-chemokines with their receptors (11 12). We also altered any residues that differed between human CCR5 and its murine homologue (which is non-functional for HIV-1 entry) (9, 10) whenever the difference involved a charge change. In all, 15 single, four double and one triple mutants were tested in these studies (FIG. 4).

The wt and mutant CCR5 proteins (HA-tagged at the C-terminus to facilitate detection) were transiently expressed in both U87MG-CD4 and SCL-1-CD4 cells and their abilities to support entry mediated by HIV-1 envelope glycoproteins were determined using an env-complementation assay with a luciferase readout (FIG. 5) (1, 2). These non-lymphoid human cell lines were chosen because they lack the CCR5, CCR3 and CXCR4 co-receptors, so resist infection by HIV-1 in the absence of a transfected co-receptor (2, 4, 13, 14) (a few exceptional HIV-1 strains will enter U87MG-CD4 cells via an unknown route (15), but we did not use them). Almost identical results were obtained with both cell lines. Two M-tropic viruses (ADA and JR-FL) that use CCR5 but not CXCR4 (1-5), and one dual-tropic virus (DH123) (15) that uses both CCR5 and CXCR4 equally well (17), were used to test whether the mutant CCR5 proteins could support HIV-1 entry. The level of expression of each transfected CCR5 mutant was assessed by western blotting, and taken into account when determining co-receptor efficiency.

Of the 15 single mutations, only three had a significant inhibitory effect on the co-receptor function of CCR5 (FIG. 5). These were D2A, D11A and E18A, all located in the Nt domain of CCR5 (FIG. 4). The E18A substitution alone was sufficient to reduce CCR5 function by 15-20 fold. The double mutant D2A/D11A was less active than either of the single mutants, and the triple mutant (D2A/D1 1A/E18A) was almost completely inactive (>50-fold reduction in entry compared to wild-type) (FIG. 5A). None of the other substitutions significantly affected CCR5 function in this assay (FIG. 5B,C). Similar results were obtained with both M-tropic envelope glycoproteins, and the only difference noted with the dual-tropic DH123 envelope was a significantly increased sensitivity to the D11A substitution (FIG. 5A). Thus negatively charged residues in the CCR5 Nt have a major influence on the co-receptor function of this molecule.

To study the effects of the D2A, D11A and E18A substitutions in an independent assay of HIV-1 env function, we used a membrane fusion assay in which HeLa cells stably expressing the JR-FL env gene are mixed with HeLa-CD4 cells transiently transfected with wild-type or mutant CCR5 (FIG. 6) (1, 18). The two cell types are labeled with different fluorescent probes, and fusion is monitored by resonance energy transfer (RET), which occurs only when the two dyes are present in the same membrane (1, 18). We tested the D2A, D11A and E18A single mutants and the double and triple mutants, in comparison to wild type CCR5 in the RET assay. Each mutant had a phenotype in this fusion assay identical to what was observed in the viral entry assay (cf. FIGS. 5A,6); the E18A and the double and triple mutants were completely unable to support env-mediated membrane fusion. However, when we boosted the expression of coreceptors by about 100-fold using the vaccinia-T7 polymerase (vFT7-pol) system (1, 4.5, 13), each of the CCR5 mutants was able to support membrane fusion, although less efficiently than the wild-type protein (FIG. 6). We noted previously that CCR5 over-expression abolished the ability of its CC-chemokine ligands to inhibit membrane fusion, suggesting that some phenotypic changes can be missed if CCR5 expression is too high (1). The results with the vac-T7pol system do, however, show that even the triple mutant (D2A/D1 1A/E18A) is not completely inert as a co-receptor, just very strongly impaired.

We next tested whether the CCR5 mutants that supported HIV-1 entry were sensitive to the inhibitory effects of the CC-chemokine ligands of CCR5: MIP-1alpha, MIP-1β and RANTES (Table 1) (19-21)

TABLE 6

| | | MIP-1α | MIP-1β | RANTES |
|---|---|---|---|---|
| | | Wt | | |
| | | 100 | 100 | 100 |
| Nt | D2A | 81 | 97 | 85 |
| | D11A | 10 | 41 | 7 |
| | N13A | 121 | 93 | 92 |
| | E18A | 100 | 100 | 62 |
| | K22A | 19 | 12 | −11 |
| | K26A | 100 | 97 | 100 |
| | R31A | −5 | 2 | −16 |
| ECL1 | Q93A | 88 | 97 | 114 |
| | D95A | 107 | 112 | 121 |
| | Q102A | 107 | 98 | 93 |
| ECL2 | K171A/E172A | 21 | 97 | 107 |
| | H175A | 119 | 100 | 95 |
| | H181A | 29 | 53 | 39 |
| | Y184A | 102 | 68 | 36 |
| | Q188A | 95 | 75 | 51 |
| | K191A/N192A | 14 | 15 | 18 |
| ECL3 | E262A | 100 | 102 | 100 |
| | R274A/D276A | 48 | 36 | 33 |

Table 6: Inhibition of co-receptor function by CC-chemokines

U87MG-CD4 cells were transiently lipofected with wild-type or mutant CCR5, then infected with NLluc/JR-FL, in the presence or absence of 2 μg/ml of MIP-1α, MIP-1β or RANTES. Luciferase activity was measured 72 h later (1.2). The relative percent inhibition by a CC-chemokine for each mutant is defined as [1-(luciferase c.p.s with chemokine/luciferase c.p.s. without chemokine)]/[1−(wild-type luciferase c.p.s with chemokine/wild-type luciferase c.p.s. without chemokine)]×100%. Each value is a mean of 3 independent experiments, each performed in quadruplicate. Mutant co-receptors for which the relative percent inhibition is <50% of that observed with wild-type CCR5 are shaded.

(Note that although the D2A, D11A and E18A mutants, are impaired for HIV-1 entry, they did support enough entry for the sensitivity to inhibition to be determined. However, this was not true of the Nt double and triple mutants). In U87MG-CD4 cells, as with other non-lymphoid cells (1, 2, 22), the CC-chemokines do not completely block HIV-1 infection, and high concentrations are needed to obtain an effect. Thus we compared the degree of inhibition achieved by the CC-chemokines on the mutant and wild-type CCR5 receptors (40-60%, depending on the particular ligand, with individual potency being RANTES>MIP-1β>MIP-1α). The following mutants were relatively insensitive to the action of one or more of the CC-chemokines: D11A, K22A, R31A (Nt), H181A, Y184A, K171A/E172A, K191A/N192A (ECL2), R274A/D276A (ECL3). Of these, only D11A was impaired for both HIV-1 entry and CC-chemokine inhibition of entry. Amino acid substitutions at certain positions (mostly in the Nt and ECL2) do not, therefore, affect the HIV-1 co-receptor function of CCR5, but do affect CC-chemokine-mediated inhibition of this process (Table 6). The way in which these substitutions affect the action of the CC-chemokines has not yet been determined. However, the simplest interpretation is that the CC-chemokine binding site and the HIV-1 interactive site on CCR5 are not identical, and that certain substitutions in ECL2 and ECL3 affect only the CC-chemokine binding site.

To understand how the Nt substitutions affect the HIV-1 co-receptor function of CCR5, we determined whether they affected gp120 binding. We were unable to measure the binding of labeled gp120 to CCR5 directly, because the level of CCR5 expression on transiently transfected cells was too low to obtain a reproducible signal in any of several binding assays tested. We therefore used a competition assay, in which the ability of gp120 (JR-FL) (7) to inhibit the binding of a phycoerythrin (PE)-labeled CCR5-specific MAb (2D7-PE) (23-25) was measured. The epitope for this MAb is located within ECL2, and we found that it was able to bind efficiently to HeLa cells co-transfected with CD4 and the CCR5 Nt mutants.

Independent studies show that 2D7 inhibits the binding of (125) I-labeled gp120 to CCR5 on the murine L1.2 cell line (25), which overexpresses CCR5 to an extent that permits the detection of gp120 binding (6, 25). Here we show that the binding of 2D7-PE to wild type CCR5 was strongly inhibited (70%) by prior addition of gp120, indicating that the interaction of gp120 and 2D7 with the receptor is mutually exclusive (FIG. 7). However, gp120 only partially inhibited (40%) the binding of 2D7-PE to the D2A, D11A and E18A mutants, and was almost ineffective at blocking 2D7-PE binding to the double and triple Nt mutants (25% and 15% inhibition, respectively) (FIG. 7). Of note, those mutants most impaired for HIV-1 entry (FIG. 5) were also the ones for which 2D7-PE binding was least sensitive to gp120 inhibition (FIG. 7). The most probable explanation of this result is that gp120 binds to the wild type CCR5 molecule in such a way as to sterically hinder binding of 2D7-PE to ECL2, but binds poorly to the Nt mutants. A less likely possibility is that gp120 does bind efficiently to the Nt mutants but in an unusual orientation in which it is less able to inhibit 2D7-PE binding to ECL2. In the latter case, the geometry of inter-domain interactions in CCR5 has been altered by the Nt substitutions that impair CCR5 co-receptor function.

In this study, we have identified point substitutions at three negatively-charged residues in the amino-terminal domain that affect the co-receptor function of CCR5, without necessarily interfering with CC-chemokine inhibition of co-receptor function. The same substitutions affect the ability of gp120 to interact correctly with CCR5, probably by reducing the affinity of the gp120-CCR5 interaction. This may be sufficient to account for the co-receptor-defective phenotype. The loss of affinity for gp120 caused by the Nt substitutions in CCR5 can be partially compensated for by overexpressing the mutant co-receptors (FIG. 6), presumably because an increase in the number of low affinity co-receptors enables a successful gp120-CCR5 interaction to occur sufficiently rapidly to be compatible with the conformational changes in the envelope glycoproteins that initiate membrane fusion (26-28).

The gp120 binding site on CCR5 is therefore dependent on residues in the Nt, and it is possible that a discrete gp120-binding domain is actually confined to the Nt. Previous studies using chimeric receptors or deletion mutants indicated the importance of the CCR5 and CXCR4 Nt's for co-receptor function (8-10, 29). The chimera studies also suggested that the site of interaction between CCR5 and HIV-1 is relatively broad and somewhat flexible (8-10). Although this possibility should not be discounted, alterations in the extracellular loops of receptor chimeras may also indirectly affect the orientation of the CCR5 Nt and hence its ability to interact correctly with gp120. In contrast to the gp120-binding site, the CC-chemokine binding site on CCR5 is dependent on residues in both the Nt and the extracellular loops (notably, but not exclusively, ECL2). Thus, although there is some overlap between the gp120 and CC-chemokine binding sites (as indicated by gp120 inhibition of CC-chemokine binding to CCR5) (6.7) they are not identical, a conclusion consistent with studies showing that signal transduction and co-receptor activity are separable functions of CCR5 (10, 30, 31).

A more detailed understanding of the interactive sites on CCR5 for gp120 and the CC-chemokines (and on these molecules for CCR5) will be required to define how HIV-1 uses CCR5 for entry into its target cells and, perhaps, for the development of inhibitors of this process. It will also be important to determine whether the negatively charged residues that we have identified in the CCR5 Nt interact directly or indirectly with positively charged amino acids in gp120, in the V3 loop and/or elsewhere.

Methods

Lipofections and reporter gene assays Mutated cDNAs were subcloned into the pcDNA3.1 (Stratagene) expression vector. U87MG-CD4 and SCL-1-CD4 cells were incubated with lipofectin (5 μg/ml) and pSVlacZ (5 μg/ml), or mutant DNA (4 μg/ml)+pSVlacZ (1 μg/ml) in OPTI-MEM (Gibco BRL), for 5 h at 37° C. The cells were infected 24 h later with NLluc/Env supernatants, containing 200-500 ng/ml p24, for 2 h at 37° C. For CC-chemokine blocking of HIV-1 entry, 2 μg/ml of MIP-1α, MIP-1β or RANTES (R & D Systems) was added simultaneously with HIV-1 (50-100 ng/ml p24), and maintained in the cultures for 12 h. Cell samples were treated with 100 μl of lysis buffer (Promega) 72 h after infection, and luciferase (luc) and β-galactosidase activity (OD₄₂₀) were measured (1, 2). Standardized luciferase activity is defined as (luc c.p.s/ng/ml p24/(OD₄₂₀+control OD₄₂₀)/(r.f.e. for mutant CCR5 bands÷r.f.e. for wild-type CCR5 bands) (see below). Luc c.p.s. values ranged from 5×10⁵ to 2×10⁶ for wild-type CCR5.

Immunoblot analysis of CCR5 expression in whole cell extracts All CCR5 molecules used in this study had a 9-residue hemagglutinin (HA)-tag as a C-terminal extension, to facilitate detection. Lipofected U87MG-CD4 cells from a 60 mm tissue culture plate were resuspended in 1% sodium dodecyl maltoside, 10 mM Tris-HCl (pH 6.8), 50 mM NaCl, 1 mM CaCl₂₁ 0.1 mM PMSF, 5 μg/ml leupeptin, 10 μg/ml aprotinin, 0.7 μg/ml pepstatin and 10 mM EDTA. The suspension was incubated at 4° C. for 30 min and the supernatant fraction collected. Total protein concentration was determined using the Bio-Rad DC Protein Assay. Protein (15 μg) was then fractionated, without prior boiling, on an SDS-polyacrylamide gel. Proteins were transferred to Immobilon-P membranes (Millipore) and probed for CCR5 with rabbit anti-HA-tag antibody (1:500 dilution; Berkeley Antibody Company) and AP-labeled goat anti-rabbit IgG (1:10⁴ dilution; Amersham), followed by incubation with chemifluorescent substrate (Vistra ECF, Amersham). Relative fluorescence emission (r.f.e.) of immunoreactive bands, excited at 450 nm, was detected on a laser-based scanner (Molecular Dynamics Storm 860). Identical expression patterns were obtained with whole-cell extracts and plasma-membrane extracts. CCR5 mutant expression levels varied from 20% to 100% of the wild-type protein. The relationship between wild-type CCR5 expression levels and HIV-1 entry efficiency was determined to be linear over a 10-fold range (data not shown).

Competition between gp120 and 2D7 MAb for CCR5 binding HeLa cells (2×10⁶) were incubated for 5 h with lipofectin (10 μg/ml) and the pCDM8 CD4 expression vector (3.75 μg/ml) plus wild-type or mutant CCR5 plasmids (1.25 μg/ml) in OPTI-MEM. The cells were then infected for 12 h with 2×10⁷ p.f.u. of vFT7 to boost CCR5 expression (1), detached with 2 mM EDTA/PBS, and washed once with binding buffer (1% BSA, 0.05% azide in DPBS). Cells (1×10⁶) were incubated for 1 h at 37° C. with or without 10 g/ml gp120 (JR-FL) (7) before addition of PE-labeled 2D7 MAb (23, 24) (20 ng/ml) for 30 min at 4° C. The cells were washed once with binding buffer and once with PBS, resuspended in 1% formaldehyde/PBS and analyzed by FACS to determine mean fluorescence intensity (m.f.i.). CD4 expression was monitored by staining with Leu3A, and varied by no more than ±10% between samples. CCR5 mutant expression levels ranged from 20% to 100% of that of wild type CCR5.

### REFERENCES OF THE THIRD SERIES OF EXPERIMENTS

1. Dragic, T., et al. HIV-1 entry into CD4+ cells is mediated by the chemokine receptor CC-CKR-5. Nature 381, 667-673 (1996).
2. Deng, H. K., et al. Identification of a major co-receptor for primary isolates of HIV-1. Nature 381, 661-666 (1996).
3. Choe, H., et al. The β-chemokine receptors CCR3 and CCR5 facilitate infection by primary HIV-1 isolates. Cell 86, 1135-1148 (1996).
4. Doranz, B. J., et al. A dual-tropic, primary HIV-1 isolate that uses fusin and the β-chemokine receptors CKR-5, CKR-3, and CKR-2b as fusion cofactors. Cell 86, 1149-1159 (1996).

5. Alkhatib, G., et al. CC CKR5: A RANTES, MIP-1α, MIP-1β receptor as a fusion cofactor for macrophage-tropic HIV-1. Science 272, 1955-1958 (1996).
6. Wu, L., et al. CD4-induced interaction of primary HIV-1 gp120 glycoproteins with the chemokine receptor CCR-5. Nature 384, 179-183 (1996).
7. Trkola, A., et al. CD4-dependent, antibody-sensitive interactions between HIV-1 and its co-receptor CCR5. Nature 384, 184-186 (1996).
8. Rucker, J., et al. Regions in β-chemokine receptors CCR5 and CCR2b that determine HIV-1 cofactor specificity. Cell 87, 437-446 (1996).
9. Atchison, R. E., et al. Multiple extracellular elements of CCR5 and HIV-1 entry: Dissociation from response to chemokines. Science 274, 1924-1926 (1996).
10. Bieniasz, P. D., et al. HIV-1 induced cell fusion is mediated by multiple regions within both the viral envelope and the CCR-5 co-receptor. EMBO J. (in press).
11. Murphy, P. The molecular biology of leukocyte chemoattractant receptors. Annu. Rev. Immunol. 12, 593-633 (1994).
12. Wells, T. N. C., et al. Selectivity and antagonism of chemokine receptors. J. Leukoc. Biol. 59, 53-60 (1996).
13. Feng, Y., Broder, C. C., Kennedy, P. E. & Berger, E. A. HIV-1 entry cofactor: Functional cDNA cloning of a seven-transmembrane G protein-coupled receptor. Science 272, 872-877 (1996).
14. Berson, J. F., et al. A seven transmembrane domain receptor involved in fusion and entry of T-cell tropic human immunodeficiency virus type-1 strains. J. Virol. 70, 6288-6295 (1996).
15. McKnight, A., et al. Inhibition of human immunodeficiency virus fusion by a monoclonal antibody to a coreceptor (CXCR4) is both cell type and virus strain dependent. J. Virol. 71, 1692-1696 (1997).
16. Shibata, R. et al. Isolation and characterization of a syncytium-inducing, macrophage/T-cell line-tropic human immunodeficiency virus type 1 isolate that readily infects chimpanzee cells in vitro and in vivo. J. Virol. 69, 4453-4462 (1995).
17. Trkola, A., Dragic, T. & Moore, J. P. (unpublished results).
18. Litwin, V. et al. Human immunodeficiency virus type 1 membrane fusion mediated by a laboratory-adapted strain and a primary isolate analyzed by resonance energy transfer. J. Virol. 70, 6437-6441 (1996).
19. Cocchi, F., et al. Identification of RANTES, MIP-1 alpha and MIP-1 beta as the major HIV suppressive factors produced by CD8+ T cells. Science 270, 1811-1815 (1995).
20. Samson, M., Labbe, O., Mollereau, C., Vassart, G. & Parmentier, M. Molecular cloning and functional expression of a new CC-chemokine receptor gene, CC-CKR5. Biochemistry 11, 3362-3367 (1996).
21. Raport, C. J., Gosling, J., Schweickart, V. L., Gray, P. W. & Charo, l. F. Molecular cloning and functional characterization of a novel human CC-chemokine receptor (CCR5) for Rantes, MlP-1beta, and MlP-1alpha. J. Biol. Chem. 271, 17161-17166 (1996).
22. Moriuchi, M., Moriuchi, H., Combadiere, C., Murphy, P. M. & Fauci, A. S. CD8+ T cell-derived factor(s), but not β-chemokines RANTES, MIP-1α, and MIP-1β, suppress HIV-1 replication in monocyte/macrophages. Proc. Natl. Acad. Sci. USA 93, 15341-15345 (1996).
23. Bleul, C. C., Wu, L., Hoxie, J. A., Springer, T. A., & Mackay, C. R. The HIV coreceptors CXCR4 and CCR5 are differentially expressed and regulated on human T Iymphocytes. Proc. Natl. Acad. Sci. USA 94, 1925-1930 (1997).
24. Wu, L., et al. CCR5 levels and expression pattern correlate with infectability by macrophage tropic HIV-1, in vitro. J. Exp. Med. 185, 1681-1691 (1997).
25. Wu, L. et al. (Manuscript in preparation).
26. Chan, D. C., Fass, D., Berger, J. M. & Kim, P. S. Core structure of gp41 from the HIV envelope glycoprotein. Cell 89, 263-273 (1997).
27. Weissenhorn, W., Dessen, A., Harrison, S. C., Skehel, J. J. & Wiley, D. C. Atomic structure of the ectodomain from HIV-1 gp41. Nature 387, 426-430 (1997).
28. Binley, J. & Moore, J. P. HlV-cell fusion. The viral mousetrap. Nature 387, 346-348 (1997).
29. Picard, L. et al. Role of the amino-terminal extracellular domain of CXCR-4 in human immunodeficiency virus type 1 entry. Virology 231, 105-111 (1997).
30. Farzan, M., et al. HIV-1 entry and macrophage inflammatory protein-1β-mediated signaling are independent functions of the chemokine receptor CCR5. J. Biol. Chem. 272, 6854-6857 (1997).
31. Gosling, J. et al. Molecular uncoupling of C-C chemokine receptor 5-induced chemotaxis and signal transduction from HIV-1 coreceptor activity. Proc. Natl. Acad. Sci. USA 94, 5061-5066 (1997).
   Fourth Series of Experiments
   Direct Binding of HIV-1_{JR-FL} gp120 to CCR5⁺ CD4⁻ Cells

The direct binding of HIV-1_{JR-FL} gp120 to CCR5⁺ CD4⁻ cells has been demonstrated. In this case, preincubation of the gp120 with sCD4 or another CD4-based molecule is required, presumably because this results in a conformational change in gp120 that exposes a chemokine receptor binding site. FIG. 8 illustrates the use of flow cytometry to measure the direct binding of sCD4/gp120 complexes to human CCR5-bearing murine L1.2 cells. Background levels of binding were observed with either biotinylated protein alone, or if gp120 from the laboratory-adapted strain HIV-1 μm is used in place of the HIV-1_{JR-FL} gp120 (data not shown).

This assay has been adapted for drug screening purposes to a 96-well microplate format where binding of the sCD4/gp120 complexes to CCR5⁺/CD4⁻ cells is measured using a fluorometric plate reader. One method is as follows:
1) Plate out L1.2-CCR5⁺ cells (approx. 500,000/well).
2) Add inhibitor for 1 hour at room temperature.
3) Wash and add biotinylated sCD4 (2.5 μg/ml) and biotinylated HIV-1_{JR-FL} gp120 (5 μg/ml), then incubate for 2 hours at room temperature.
4) Wash and incubate with streptavidin-phycoerythrin (100 ng/nl).
5) Wash and measure the amount of bound gp120/sCD4 using a fluorometric plate reader exciting at 530 nm and reading emission at 590 nm.

Using this method, inhibition of binding of gp120/sCD4 to CCR5 by CC-chemokines (FIG. 9) and antibodies to CCR5 that block HIV-1 infection (not shown) have been demonstrated.

Inhibition of HIV-1 Envelope-Mediated Membrane Fusion by Extracellular Domains of CCR5.

Synthetic peptides representing the four extracellular domains of human CCR5 were made by Quality Controlled Biochemicals (Hopkinton, Mass.) and tested for ability to inhibit membrane fusion mediated by the envelope glycoproteins of the LAI or JR-FL strains of HIV-1 using the resonance energy transfer (RET) assay described above. Specific inhibition of fusion mediated by the JR-FL envelope glycoprotein was seen using the ECL2 peptide but not other peptides. ECL2 inhibited fusion between HeLa-env_{JR-FL} cells and PM1 cells by 97% at 100 μg/ml, 65% at 33 μg/ml and 15% at 11 μg/ml (mean of two assays). ECL2 gave no inhibition of fusion between HeLa-env_{LAI} and PMI cells or HeLa-env_{LAI} and HeLa-CD4 cells. These results strongly suggest that CCR5 ECL2 specifically inhibits fusion, most likely by blocking the interaction between HIV-1_{JR-FL} gp120 and CCR5. No other peptides tested gave significant levels of specific inhibition of fusion.

Inhibition of HIV-1 Envelope-Mediated Membrane Fusion by the Bicyclam, JM3100.

The bicyclam JM3100, obtained from Dr. J. Moore (Aaron Diamond AIDS Research Center, NY) was tested for ability to inhibit membrane fusion mediated by the envelope glycoproteins of the LAI or JR-FL strains of HIV-1 using the resonance energy transfer (RET) assay described above. As illustrated in FIG. 10, this molecule specifically and potently inhibits fusion mediated by gp120/gp41 from the HIV-1_{LAI} strain, and not from the HIV-1_{JR-FL} strain. These data suggest that this molecule specifically inhibits HIV fusion by blocking the interaction between HIV-1_{LAI} gp120 and CXCR4.

1-48. (canceled)

49. A polypeptide having a sequence corresponding to the sequence of a portion of a chemokine receptor and capable of inhibiting the fusion of HIV-1 to CD4⁺ cells and thus of inhibiting HIV-1 infection of the cells.

50. The polypeptide of claim 49, wherein the chemokine receptor is CCR5.

51. The polypeptide of claim 50 comprising consecutive amino acids having a sequence present in at least one extracellular domain of CCR5.

52. The polypeptide of claim 51, wherein the at least one extracellular domain is the second extracellular loop.

53. The polypeptide of any one of claims 49 to 52, wherein the portion of the chemokine receptor comprises consecutive amino acids, the amino acid sequence of which comprises a sequence shown in any of SEQ ID NO:5, SEQ ID NO:6, SEQ ID NO:7, or SEQ ID NO:8.

54. The polypeptide of claim 50, determined to be capable of inhibiting the fusion of HIV-1 to a CD4⁺ CCR5⁺ cell using a method which comprises:
(a) contacting the CD4⁺ CCR5⁺ cell, labeled with a first dye, with a cell expressing an appropriate HIV-1 envelope glycoprotein on its surface and labeled with a second dye, in the presence of an excess of the polypeptide under conditions permitting fusion of the CD4⁺ CCR5⁺ cell to the cell expressing the HIV-1 envelope glycoprotein on its surface in the absence of the polypeptide, the first and second dyes being selected so as to allow resonance energy transfer between the dyes;
(b) exposing the product of step (a) to conditions which would result in resonance energy transfer if fusion has occurred; and
(c) determining whether there is resonance energy transfer, the absence of, or reduction in, resonance energy transfer indicating that fusion has not occurred and that the polypeptide is capable of inhibiting fusion of HIV-1 to a CD4⁺ CCR5⁺ cell.

55. A pharmaceutical composition comprising the polypeptide of any of claims 49 to 54 and a pharmaceutically acceptable carrier.

56. A method for treating HIV-1 infection in a subject which comprises administering to the subject the polypeptide of any of claims 49 to 54 in an amount effective to inhibit the fusion of HIV-1 to CD4⁺ cells of the subject, so as to thereby treat the subject.

57. A method of reducing the likelihood of HIV-1 infection in a subject which comprises administering to the subject the polypeptide of any of claims 49 to 54 in an amount effective to inhibit the fusion of HIV-1 to CD4⁺ cells of the subject, so as to thereby reduce the likelihood of HIV-1 infection.

58. A method for inhibiting HIV-1 infection of CD4⁺ cells which comprises contacting the CD4⁺ cells with the polypeptide of any of claims 49 to 54 under conditions effective to inhibit the fusion of HIV-1 to CD4⁺ cells, so as to thereby inhibit HIV-1 infection of the CD4⁺ cells.
